(19) United States
(12) Patent Application Publication
(10) Pub. No.: US 2023/0399614 A1
(43) Pub. Date: Dec. 14, 2023

(54) METHODS OF PREPARING LYMPHOCYTES FOR CELL THERAPY

(71) Applicant: Kite Pharma, Inc., Foster City, CA (US)

(72) Inventors: Saikat Banerjee, Oakland, CA (US); Jodi L. Murakami, Culver City, CA (US); Heba N. Nowyhed, Los Angeles, CA (US); Da Ming Ou, Castro Valley, CA (US)

(21) Appl. No.: 18/330,962

(22) Filed: Jun. 7, 2023

Related U.S. Application Data
(60) Provisional application No. 63/350,645, filed on Jun. 9, 2022.

(51) Int. Cl. C12N 5/0783; C12N 15/86; C07K 16/28; C07K 14/725

(52) U.S. Cl. CPC C12N 5/0636; C12N 15/86; C07K 16/2809; C07K 16/2818; C07K 14/7051; C12N 2501/515; C12N 2501/51; C12N 2501/2307; C12N 2501/2321; C12N 2501/2302; C12N 2740/15043

(57) ABSTRACT

The present disclosure is directed to methods of preparing genetically engineered lymphocytes. In addition, methods of using the genetically engineered lymphocytes in T cell therapy for cancers are also disclosed.

### CROSS REFERENCE TO RELATED APPLICATIONS

This application claims the benefit of U.S. Provisional Patent Application No. 63/350,645, filed on Jun. 9, 2022, which is hereby incorporated by reference its entirety.

### FIELD

The present application relates to methods of preparing one or more lymphocytes, e.g., T cells, for cell therapy. The present application also relates to the cells that are prepared, generated, or processed using the methods disclosed herein. In certain aspects, the present application relates to a method of improving the efficacy of a cell therapy by contacting one or more lymphocytes with an anti-CD81 antibody, an exogenous Interleukin-7 (IL-7) and an exogenous Interleukin-21 (IL-21).

### BACKGROUND

Human cancers are by their nature comprised of normal cells that have undergone a genetic or epigenetic conversion to become abnormal cancer cells. In doing so, cancer cells begin to express proteins and other antigens that are distinct from those expressed by normal cells. These aberrant tumor antigens can be used by the body's innate immune system to specifically target and kill cancer cells. However, cancer cells employ various mechanisms to prevent immune cells, such as T and B lymphocytes, from successfully targeting cancer cells.

Human T cell therapies rely on ex-vivo enriched or modified human T cells to target and kill cancer cells in a subject, e.g., a patient. Various technologies have been developed to enrich the concentration of naturally occurring T cells capable of targeting a tumor antigen or genetically modifying T cells to specifically target a known cancer antigen. These therapies have proven to have promising effects on tumor size and patient survival. However, it has proven difficult to predict whether a given T cell therapy will be effective in each patient.

Transplantation of a mixed population of T cells is among the factors hindering T cell therapies from reaching their full potential. In conventional T cell therapies, donor T cells are collected, optionally modified to target a specific antigen (e.g., a tumor cell) or selected for anti-tumor characteristics (e.g., tumor infiltrating lymphocytes), expanded in vitro, and administered to a subject in need thereof. Typically, the resulting T cells comprise a mixed population of largely mature cells, many of which are terminally differentiated. As a result, the expected in vivo persistence of these cells can be limited, and positive effects initially observed can be undone over time as tumors rebound in the absence of transplanted T cells. Thus, there remains a need to increase the in vivo persistence of T cells for use in a T cell therapy.

Current T cell therapies rely on enriched or modified human T cells to target and kill cancer cells in a patient. To increase the ability of T cells to target and kill a particular cancer cell, methods have been developed to engineer T cells to express constructs which direct T cells to a particular target cancer cell. Chimeric antigen receptors (CARs) and engineered T cell receptors (TCRs), which comprise binding domains capable of interacting with a particular tumor antigen, allow T cells to target and kill cancer cells that express the particular tumor antigen.

A need exists for improved CARs and TCRs for targeting and killing cancer cells as well as improved methods for preparing lymphocytes expressing CARs and/or TCRs for use in cell therapy.

### SUMMARY OF THE DISCLOSURE

Any aspect or embodiment described herein may be combined with any other aspect or embodiment as disclosed herein. Other aspects, advantages, and modifications are within the scope of the application.

The present disclosure provides a method of manufacturing a genetically engineered lymphocyte comprising contacting in vitro one or more lymphocytes from a subject with an anti-CD81 antibody, an exogenous Interleukin-7 (IL-7) and an exogenous Interleukin-21 (IL-21), transforming the contacted lymphocyte with a vector containing a gene of interest, and harvesting the lymphocyte.

The present disclosure further provides a method of manufacturing a genetically engineered lymphocyte, wherein the lymphocyte is selected from the group consisting of macrophages, neutrophils, basophils, eosinophils, granulocytes, natural killer cells (NK cells), B cells, T cells, NK-T cells, mast cells, tumor infiltrating lymphocytes (TILs), myeloid derived suppressor cells (MDSCs), and dendritic cells.

In certain embodiments, the lymphocyte is a T cell. In some embodiments, the lymphocyte is contacted with an anti-CD3 antibody and an anti-CD28 antibody.

In certain embodiments, the T cells comprise CD8+ T cells and CD4+ T cells. In some embodiments, the CD8+ T cells express CCR7+ and/or CD45RA+. In some embodiments, the CD4+ T cells express CCR7+ and/or CD45RA+. In certain embodiments, the CD8+ T cells express CD27+ and/or CD28+. In some embodiments, the CD4+ T cells express CD27+ and/or CD28+. In certain embodiments, the CD8+ T cells express CD27+ CD28+ CCR7+ and/or CD45RA+. In some embodiments, the CD4+ T cells express CD27+ CD28+ CCR7+ and/or CD45RA+.

The present disclosure further provides a method of manufacturing a genetically engineered lymphocyte, wherein the T cells express a chimeric antigen receptor (CAR). In certain embodiments, the chimeric antigen receptor (CAR) is bicistronic. In certain embodiments, the chimeric antigen receptor (CAR) is bispecific. In some embodiments, the chimeric antigen receptor (CAR) binds to CD19. In certain embodiments, the chimeric antigen receptor (CAR) comprises a single chain variable fragment (scFv) targeting an identified tumor antigen comprising CD20, BCMA, CLL-1, CTLA4, CD30, CD40, NKp44, NKp30, GPC-3, CD79a, CD79b, BAFF-R, CS-1, PSMA, NKG2D, CLL-1, CD33, CD22 or NKp46.

The present disclosure also provides a method of manufacturing a genetically engineered lymphocyte, wherein the vector is a retroviral vector, a DNA vector, a plasmid, an RNA vector, an adenoviral vector, an adenovirus associated vector, a lentiviral vector, or any combination thereof. In certain embodiments, the DNA vector is a transposon.

The present disclosure further provides a method of manufacturing a genetically engineered lymphocyte, wherein the lymphocytes have not been contacted with an exogenous Interleukin-2 (IL-2).

In certain embodiments, a donor is a subject in need of a T cell therapy.

The present disclosure also provides a method of manufacturing a genetically engineered lymphocyte, wherein the lymphocytes are transduced with a viral vector containing the gene of interest. In certain embodiments, the viral vector is a lentiviral vector. In some embodiments, the viral vector is a retroviral vector.

In certain embodiments, the lymphocytes are harvested no more than 24 hours after the transformation. In some embodiments, the lymphocytes are harvested no more than 2 days after the transformation. In certain embodiments, the lymphocytes are harvested no more than 3 days after the transformation. In some embodiments, the lymphocytes are harvested no more than 5 days after the transformation. In certain embodiments, the lymphocytes are harvested no more than 7 days after the transformation. In some embodiments, the lymphocytes are harvested no more than 8 days after the transformation. In certain embodiments, the lymphocytes are harvested no more than 9 days after the transformation. In certain embodiments, the lymphocytes are harvested no more than 9 days after the transformation. In certain embodiments, the lymphocytes are harvested no more than 10 days, no more than 11 days, no more than 12 days, no more than 13 days or no more than 14 days after transformation.

Another aspect of the disclosure is directed to a genetically engineered lymphocyte produced by a method comprising contacting in vitro one or more lymphocytes from a subject with an anti-CD81 antibody, an exogenous Interleukin-7 (IL-7) and an exogenous Interleukin-21 (IL-21), transforming the contacted lymphocyte with a vector containing a gene of interest; and harvesting the lymphocyte. In certain embodiments, the lymphocyte is selected from the group consisting of macrophages, neutrophils, basophils, eosinophils, granulocytes, natural killer cells (NK cells), B cells, T cells, NK-T cells, mast cells, tumor infiltrating lymphocytes (TILs), myeloid derived suppressor cells (MDSCs), and dendritic cells.

In certain embodiments, the genetically engineered lymphocyte has a higher juvenile and a less differentiated CAR-T phenotype compared to the genetically engineered lymphocyte produced in contacting in vitro one or more lymphocytes from a subject with an exogenous Interleukin-2 (IL-2).

In certain embodiments, the genetically engineered lymphocyte further comprises transforming the contacted lymphocyte with a vector containing a gene of interest and harvesting the lymphocyte. In some embodiments, the lymphocyte is T cell.

In some embodiments, the higher juvenile and less differentiated CAR-T phenotype comprises CCR7+ CD45RA+ CD4+ T cells. In certain embodiments, the higher juvenile and less differentiated CAR-T phenotype comprises CCR7+ CD45RA+ CD8+ T cells.

In some embodiments, the higher juvenile and less differentiated CAR-T phenotype is produced when the lymphocytes are harvested no more than 6 days after the transformation. In certain embodiments, the higher juvenile and less differentiated CAR-T phenotype is produced when the lymphocytes are harvested no more than 8 days after the transformation. In certain embodiments, the higher juvenile and less differentiated CAR-T phenotype is produced when the lymphocytes are harvested no more than 9 days, no more than 10 days, no more than 11 days, no more than 12 days, no more than 13 days or no more than 14 days after transformation.

In certain embodiments, the genetically engineered lymphocyte secretes a lower effector cytokine compared to the genetically engineered lymphocyte produced in contacting in vitro one or more lymphocytes from a subject with an exogenous Interleukin-2 (IL-2).

In certain embodiments, the lower effector cytokine is Granzyme A. In some embodiments, the lower effector cytokine is IFN-γ. In certain embodiments, the genetically engineered lymphocytes further secrete higher IL-2.

In certain embodiments, the genetically engineered lymphocytes exhibit a more juvenile phenotype compared to the genetically engineered lymphocyte produced in contacting in vitro one or more lymphocytes from a subject with an exogenous Interleukin-2 (IL-2).

Another aspect of the disclosure is directed to a composition comprising the genetically engineered lymphocytes.

The present disclosure further provides a method of treating a cancer comprising: administering the composition to a subject in need of treatment; and monitoring the subject to determine the progress of the treatment.

Another aspect of the disclosure is directed to the use of a genetically engineered lymphocyte for the manufacture of a composition for the treatment of a cancer, wherein the genetically engineered lymphocyte is a T cell.

In certain embodiments, the genetically engineered lymphocyte is used for the treatment of a cancer.

In some embodiments, the cancer is selected from the group consisting of acute lymphoblastic leukemia (ALL), acute myeloid leukemia (AML), adenoid cystic carcinoma, adrenocortical, carcinoma, AIDS-related cancers, anal cancer, appendix cancer, astrocytomas, atypical teratoid/rhabdoid tumor, central nervous system, B-cell leukemia, lymphoma, refractory B cell malignancy or other B cell malignancies, basal cell carcinoma, bile duct cancer, bladder cancer, bone cancer, osteosarcoma and malignant fibrous histiocytoma, brain stem glioma, brain tumors, breast cancer, bronchial tumors, burkitt lymphoma, carcinoid tumors, central nervous system cancers, cervical cancer, chordoma, chronic lymphocytic leukemia (CLL), chronic myelogenous leukemia (CML), chronic myeloproliferative disorders, colon cancer, colorectal cancer, craniopharyngioma, cutaneous t-cell lymphoma, embryonal tumors, central nervous system, endometrial cancer, ependymoblastoma, ependymoma, esophageal cancer, esthesioneuroblastoma, ewing sarcoma family of tumors extracranial germ cell tumor, extragonadal germ cell tumor extrahepatic bile duct cancer, eye cancer fibrous histiocytoma of bone, malignant, and osteosarcoma, gallbladder cancer, gastric (stomach) cancer, gastrointestinal carcinoid tumor, gastrointestinal stromal tumors (GIST), soft tissue sarcoma, germ cell tumor, gestational trophoblastic tumor, glioma, hairy cell leukemia, head and neck cancer, heart cancer, hepatocellular (liver) cancer, histiocytosis, Hodgkin lymphoma, hypopharyngeal cancer, intraocular melanoma, islet cell tumors (endocrine pancreas), kaposi sarcoma, kidney cancer, langerhans cell histiocytosis, laryngeal cancer, leukemia, lip and oral cavity cancer, liver cancer (primary), lobular carcinoma in situ (LCIS), lung cancer, lymphoma, macroglobulinemia, male breast cancer, malignant fibrous histiocytoma of bone and osteosarcoma, medulloblastoma, medulloepithelioma, melanoma, merkel cell carcinoma, mesothelioma, metastatic squamous neck cancer with occult primary midline tract carcinoma involving NUT gene, mouth cancer, multiple endocrine neoplasia syndromes, multiple myeloma/plasma cell neoplasm, mycosis fungoides, myelodysplastic syndromes, myelodysplastic/myeloproliferative neoplasms, myelogenous leukemia, chronic (CML), Myeloid leukemia, acute (AML), myeloma, multiple, myeloproliferative disorders, nasal cavity and paranasal sinus cancer, nasopharyngeal cancer, neuroblastoma, non-Hodgkin lymphoma, non-small cell lung cancer, oral cancer, oral cavity cancer, oropharyngeal cancer, osteosarcoma and malignant fibrous histiocytoma of bone, ovarian cancer, pancreatic cancer, papillomatosis, paraganglioma, paranasal sinus and nasal cavity cancer, parathyroid cancer, penile cancer, pharyngeal cancer, pheochromocytoma, pineal parenchymal tumors of intermediate differentiation, pineoblastoma and supratentorial primitive neuroectodermal tumors, pituitary tumor, plasma cell neoplasm/multiple myeloma, pleuropulmonary blastoma, pregnancy and breast cancer, primary central nervous system (CNS) lymphoma, prostate cancer, rectal cancer, renal cell (kidney) cancer, renal pelvis and ureter, transitional cell cancer, retinoblastoma, rhabdomyosarcoma, salivary gland cancer, sarcoma, sézary syndrome, small cell lung cancer, small intestine cancer, soft tissue sarcoma, squamous cell carcinoma, squamous neck cancer, stomach (gastric) cancer, supratentorial primitive neuroectodermal tumors, t-cell lymphoma, cutaneous, testicular cancer, throat cancer, thymoma and thymic carcinoma, thyroid cancer, transitional cell cancer of the renal pelvis and ureter, trophoblastic tumor, ureter and renal pelvis cancer, urethral cancer, uterine cancer, uterine sarcoma, vaginal cancer, vulvar cancer, Waldenström macroglobulinemia and Wilms Tumor.

Another aspect of the disclosure is directed to a method of treating a tumor in a subject in need of a T cell therapy comprising administering to the subject one or more T cells, wherein the one or more T cells have been contacted with an anti-CD81 antibody, an exogenous Interleukin-7 (IL-7) and an exogenous Interleukin-21 (IL-21).

The present disclosure further provides a method of reducing or decreasing the size of a tumor or inhibiting growth of a tumor in a subject in need of a T cell therapy comprising administering to the subject one or more T cells, wherein the one or more T cells have been contacted with an anti-CD81 antibody, an exogenous Interleukin-7 (IL-7) and an exogenous Interleukin-21 (IL-21).

In some embodiments, the genetically engineered lymphocytes can be used in autologous cell therapy or allogeneic cell therapy.

In some embodiments, the genetically engineered lymphocytes produce more cytokines compared to a process of making genetically engineered lymphocytes in the presence of IL-2. In some embodiments, the genetically engineered lymphocytes produce more IL-2 compared to a process of making genetically engineered lymphocytes in the presence of IL-2.

In some embodiments, the genetically engineered lymphocytes are more juvenile compared to a process of making genetically engineered lymphocytes in the presence of IL-2. In some embodiments, the genetically engineered lymphocytes are more proliferative or robust compared to a process of making genetically engineered lymphocytes in the presence of IL-2.

### DETAILED DESCRIPTION

The present disclosure relates to methods for manufacturing genetically engineered lymphocytes for use in a T cell therapy. In particular, the present disclosure relates to methods for manufacturing CAR T cells for use in a T cell therapy.

### Definitions

In order that the present disclosure can be more readily understood, certain terms are first defined. As used in this application, except as otherwise expressly provided herein, each of the following terms shall have the meaning set forth below. Additional definitions are set forth throughout the application.

Unless defined otherwise, all technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; the Dictionary of Cell and Molecular Biology, 3rd ed., 1999, Academic Press; and the Oxford Dictionary of Biochemistry and Molecular Biology, Revised, 2000, Oxford University Press, provide one of skill with a general dictionary of many of the terms used in this disclosure.

Units, prefixes, and symbols are denoted in their Système International de Unites (SI) accepted form. Numeric ranges are inclusive of the numbers defining the range. The headings provided herein are not limitations of the various aspects of the disclosure, which can be had by reference to the specification as a whole. Accordingly, the terms defined immediately below are more fully defined by reference to the specification in its entirety.

As used herein, the indefinite articles “a” or “an” should be understood to refer to “one or more” of any recited or enumerated component.

As used herein, the term “about” refers to an approximately +/−10% variation from a given value. When particular values or compositions are provided in the application and claims, unless otherwise stated, the meaning of “about” should be assumed to be within an acceptable error range for that particular value or composition.

As described herein, any concentration range, percentage range, ratio range or integer range is to be understood to include the value of any integer within the recited range and, when appropriate, fractions thereof (such as one-tenth and one-hundredth of an integer), unless otherwise indicated.

The term “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

The term “activation” or “activated” refers to the state of an lymphocyte, e.g., a T cell, that has been sufficiently stimulated to induce detectable cellular proliferation. Activation can also be associated with induced cytokine production and detectable effector functions. The term “activated T cells” refers to, among other things, T cells that are undergoing cell division. T cell activation can be characterized by increased T cell expression of one or more biomarker, including, but not limited to, CD57, PD1, CD107a, CD25, CD137, CD69, TIM-3, LAG-3, CD39 and/or CD71.

As used herein, the phrase “administering” refers to the physical introduction of an agent to a subject, using any of the various methods and delivery systems known to those skilled in the art. Exemplary routes of administration for the T cells prepared by the methods disclosed herein include intravenous, intramuscular, subcutaneous, intraperitoneal, spinal or other parenteral routes of administration, for example by injection or infusion. The phrase “parenteral administration” as used herein means modes of administration other than enteral and topical administration, usually by injection, and includes, without limitation, intravenous, intramuscular, intraarterial, intrathecal, intralymphatic, intralesional, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion, as well as in vivo electroporation. In some embodiments, the T cells prepared by the present methods is administered via a non-parenteral route, e.g., orally. Other non-parenteral routes include a topical, epidermal or mucosal route of administration, for example, intranasally, vaginally, rectally, sublingually or topically. Administering can also be performed, for example, once, a plurality of times, and/or over one or more extended periods.

The term “antibody” (Ab) includes, without limitation, an immunoglobulin which binds specifically to an antigen. In general, an antibody can comprise at least two heavy (H) chains and two light (L) chains interconnected by disulfide bonds. Each H chain comprises a heavy chain variable region (abbreviated herein as VH) and a heavy chain constant region. The heavy chain constant region can comprise three or four constant domains, CH1, CH2 CH3, and/or CH4. Each light chain comprises a light chain variable region (abbreviated herein as VL) and a light chain constant region. The light chain constant region can comprise one constant domain, CL. The VH and VL regions can be further subdivided into regions of hypervariability, termed complementarity determining regions (CDRs), interspersed with regions that are more conserved, termed framework regions (FR). Each VH and VL comprises three CDRs and four FRs, arranged from amino-terminus to carboxy-terminus in the following order: FR1, CDR1, FR2, CDR2, FR3, CDR3, FR4. The variable regions of the heavy and light chains contain a binding domain that interacts with an antigen.

An immunoglobulin can derive from any of the commonly known isotypes, including but not limited to IgA, secretory IgA, IgG and IgM. IgG subclasses are also well known to those in the art and include but are not limited to human IgG1, IgG2, IgG3 and IgG4. “Isotype” refers to the Ab (antibody) class or subclass (e.g., IgM or IgG1) that is encoded by the heavy chain constant region genes. The term “antibody” includes, by way of example, both naturally occurring and non-naturally occurring Abs; monoclonal and polyclonal Abs; chimeric and humanized Abs; human or nonhuman Abs; wholly synthetic Abs; and single chain Abs. A nonhuman Ab can be humanized by recombinant methods to reduce its immunogenicity in man. Where not expressly stated, and unless the context indicates otherwise, the term “antibody” also includes an antigen-binding fragment or an antigen-binding portion of any of the aforementioned immunoglobulins, and includes a monovalent and a divalent fragment or portion, and a single chain Ab.

An “antigen binding molecule” or “antibody fragment” refers to any portion of an antibody less than the whole. An antigen binding molecule can include the antigenic complementarity determining regions (CDRs). Examples of antibody fragments include, but are not limited to, Fab, Fab′, F(ab′)2, and Fv fragments, dAb, linear antibodies, scFv antibodies, and multispecific antibodies formed from antigen binding molecules.

The term “autologous” refers to any material derived from the same individual to which it is later to be reintroduced. For example, the engineered autologous cell therapy (eACT™) method described herein involves collection of lymphocytes from a donor, e.g., a patient, which are then engineered to express, e.g., a CAR construct, and then administered back to the same donor, e.g., patient.

The term “allogeneic” refers to any material derived from one individual which is then introduced to another individual of the same species, e.g., allogeneic T cell transplantation.

A “cancer” refers to a broad group of various diseases characterized by the uncontrolled growth of abnormal cells in the body. Unregulated cell division and growth results in the formation of malignant tumors that invade neighboring tissues and can also metastasize to distant parts of the body through the lymphatic system or bloodstream. A “cancer” or “cancer tissue” can include a tumor at various stages. In certain embodiments, the cancer or tumor is stage 0, such that, e.g., the cancer or tumor is very early in development and has not metastasized. In some embodiments, the cancer or tumor is stage I, such that, e.g., the cancer or tumor is relatively small in size, has not spread into nearby tissue, and has not metastasized. In other embodiments, the cancer or tumor is stage II or stage III, such that, e.g., the cancer or tumor is larger than in stage 0 or stage I, and it has grown into neighboring tissues, but it has not metastasized, except potentially to the lymph nodes. In other embodiments, the cancer or tumor is stage IV, such that, e.g., the cancer or tumor has metastasized. Stage IV can also be referred to as advanced or metastatic cancer.

An “anti-tumor effect” as used herein, refers to a biological effect that can present as a decrease in tumor volume, an inhibition of tumor growth, a decrease in the number of tumor cells, a decrease in tumor cell proliferation, a decrease in the number of metastases, an increase in overall or progression-free survival, an increase in life expectancy, or amelioration of various physiological symptoms associated with the tumor. An anti-tumor effect can also refer to the prevention of the occurrence of a tumor, e.g., a vaccine.

The term “progression-free survival,” which can be abbreviated as PFS, as used herein refers to the time from the treatment date to the date of disease progression per the revised IWG Response Criteria for Malignant Lymphoma or death from any cause.

“Disease progression” is assessed by measurement of malignant lesions on radiographs or other methods should not be reported as adverse events. Death due to disease progression in the absence of signs and symptoms should be reported as the primary tumor type (e.g., DLBCL).

The “duration of response,” which can be abbreviated as DOR, as used herein refers to the period of time between a subject's first objective response to the date of confirmed disease progression, per the revised IWG Response Criteria for Malignant Lymphoma, or death.

The term “overall survival,” which can be abbreviated as OS, is defined as the time from the date of treatment to the date of death.

A “cytokine,” as used herein, refers to a non-antibody protein that can be released by lymphocytes, including macrophages, B cells, T cells, and mast cells to propagate an immune response. In some embodiments, one or more cytokines are released in response to the T cell therapy. In certain embodiments, those cytokines secreted in response to the T cell therapy can be a sign of effective T cell therapy.

A “therapeutically effective amount” or “therapeutically effective dosage,” as used herein, refers to an amount of the T cells that are produced by the present methods and that, when used alone or in combination with another therapeutic agent, protects a subject against the onset of a disease or promotes disease regression evidenced by a decrease in severity of disease symptoms, an increase in frequency and duration of disease symptom-free periods, or a prevention of impairment or disability due to the disease affliction. The ability of the T cells to promote disease regression can be evaluated using a variety of methods known to the skilled practitioner, such as in human subjects during clinical trials, in animal model systems predictive of efficacy in humans, or by assaying the activity of the agent in in vitro assays.

The term “lymphocyte,” as used herein can include natural killer (NK) cells, T cells, or B cells. NK cells are a type of cytotoxic (cell toxic) lymphocyte that represent a major component of the inherent immune system. NK cells reject tumors and cells infected by viruses. It works through the process of apoptosis or programmed cell death. They were termed “natural killers” because they do not require activation in order to kill cells. T-cells play a major role in cell mediated-immunity (no antibody involvement). Its T-cell receptors (TCR) differentiate themselves from other lymphocyte types. The thymus, a specialized organ of the immune system, is primarily responsible for the T cell's maturation. The terms “immune cells” and “lymphocytes” are used interchangeably herein. There are several types of “lymphocytes” including, without limitation, macrophages (e.g, tumor associated macrophages) neutrophils, basophils, eosinophils, granulocytes, natural killer cells (NK cells), B cells, T cells, NK-T cells, mast cells, tumor infiltrating lymphocytes (TILs), myeloid derived suppressor cells (MDSCs), and dendritic cells. The term also includes precursors of these lymphocytes. Hematopoietic stem and/or progenitor cells may be derived from bone marrow, umbilical cord blood, adult peripheral blood after cytokine mobilization, and the like, by methods known in the art. Some precursor cells are those that may differentiate into the lymphoid lineage, for example, hematopoietic stem cells or progenitor cells of the lymphoid lineage. Additional examples of lymphocytes that may be used for immune therapy are described in US Publication No. 20180273601, incorporated herein by reference in its entirety.

There are several types of T-cells, namely: Helper T-cells (e.g., CD4+ cells, effector TEFF (“Effector T-cells”), Cytotoxic T cells (also known as TC, cytotoxic T lymphocyte, CTL, T-killer cell, cytolytic T cell, CD8+ T-cells or killer T cell), Memory T-cells ((i) stem memory TSCM cells, like naive cells, are CD45RO−, CCR7+, CD45RA+, CD62L+ (L-selectin), CD27+, CD28+ and IL-7Rα+, but they also express large amounts of CD95+, IL-2Pβ, CXCR3+, and LFA−, and show numerous functional attributes distinctive of memory cells); (ii) central memory TCM cells express L-selectin and are CCR7+ and CD45RO+ and they secrete IL-2, but not IFNγ or IL-4, and (iii) effector memory TEM cells, however, do not express L-selectin or CCR7 but do express CD45RO and produce effector cytokines like IPNγ and IL-4), Regulatory T-cells (Tregs, suppressor T cells, or CD4+ CD25+ regulatory T cells), Natural Killer T-cells (NKT), and Gamma Delta T-cells. T cells found within tumors are referred to as “tumor infiltrating lymphocytes” or “TIL.” B-cells, on the other hand, play a principal role in humoral immunity (with antibody involvement). It makes antibodies and antigens and performs the role of antigen presenting cells (APCs) and turns into memory B-cells after activation by antigen interaction. In mammals, immature B cells are formed in the bone marrow, where its name is derived from.

A “naive” T cell refers to a mature T cell that remains immunologically undifferentiated. Following positive and negative selection in the thymus, T cells emerge as either CD4+ or CD8+ naive T cells. In their naive state, T cells express L-selectin (CD62L+), IL-7 receptor-a (IL-7R-α), and CD132, but they do not express CD25, CD44, CD69, or CD45RO. As used herein, “immature” can also refers to a T cell which exhibits a phenotype characteristic of either a naive T cell or an immature T cell, such as a TSCM cell or a TCM cell. For example, an immature T cell can express one or more of L-selectin (CD62L+), IL-7Rα, CD132, CCR7, CD45RA, CD45RO, CD27, CD28, CD95, IL-2Rβ, CXCR3, and LFA-1. Naive or immature T cells can be contrasted with terminal differentiated effector T cells, such as TEM cells and TEFF cells.

Naive T cells, upon antigen encounter, undergo activation and differentiate into T SCM (stem memory T-cell), T CM (central memory T-cell), and effector T cells. This differentiation is marked by the expression of various cell surface markers and transcription factors along with profound alterations in cellular metabolic pathways. In general, T cell activation and differentiation is characterized by increased reliance on glycolysis and mitochondrial membrane potential. These metabolic pathways are critical to mediate effector function of T cells responding to infection and cancer. Naive T-cells are T-cells that have matured but that have not encountered an antigen. In addition to being CCR7+, CD45RO−, and CD95−, other common markers for naive T-cells are CD45RA+, IL-2Rβ−, (L-selectin), CD27+, CD28+, IL-7Rα+ and CD62L+. Stem memory T-cells (T SCM) have been described in mice, non-human primates and in humans, constituting approximately 2-4% of the total CD4+ and CD8+ T-cell population in the periphery. T SCMs represent the earliest and long-lasting developmental stage of memory T-cells, displaying stem cell-like properties, and exhibiting a gene profile between naive and central memory T-cells. In addition to being CCR7+, CD45RO−, and CD95+, other common markers for stem memory T-cells (T SCM) are CD45RA+, IL-2Rβ+, CD62L+, (L-selectin), CD27+, CD28+, IL-7Rα+, IL-2Rβ+, CXCR3+, and LFA−. Central memory T-cells (T CM) express CD45RO+, CCR7+, and CD62L+. This memory subpopulation is commonly found in the lymph nodes and in the peripheral circulation. Other common markers for central memory T-cells (T CM) are CD95+, IL-2Rβ+, CD3+, CD28+, CD127+, and granzyme B−. Effector memory T-cells (T EM) express CD45RO but lack expression of CCR7. Because these memory T-cells lack the CCR7 lymph node-homing receptors they are found in the peripheral circulation and tissues. Other common markers for effector memory T-cells (T EM) are CD95+, IL-2Rβ+, CD45RA− and CD62L−. Effector T-cells (T EFF) include cytotoxic T-cells, helper T-cells, and regulatory T-cells. In addition to being CCR7− and CD45RO−, other common markers for effector T-cells (T EFF) are CD95+, IL-2Rβ+, CD62L−, CD28−, CD62L−, CD 127−, granzyme B+, and perforin+.

“T cell function,” as referred to herein, refers to normal characteristics of healthy T cells. In some embodiments, a T cell function comprises T cell proliferation. In some embodiments, a T cell function comprises a T cell activity. In some embodiments, the T cell function comprises cytolytic activity.

Cell “proliferation” or “cell expansion,” as used herein, refers to the ability of T cells to grow in numbers through cell division. Proliferation can be measured by staining cells with carboxy fluorescein succinimidyl ester (CFSE). Cell proliferation can occur in vitro, e.g., during T cell culture, or in vivo, e.g., following administration of a T cell therapy.

“T cell activity,” as used herein, refers to any activity common to healthy T cells. In some embodiments, the T cell activity comprises cytokine production. In certain embodiments, the T cell activity comprises production of one or more cytokine selected from interferon gamma (IFNg), tissue necrosis factor alpha (TNFa), and both.

A “cytolytic activity” or “cytotoxicity,” as used herein, refers to the ability of a T cell to destroy a target cell. In some embodiments, the target cell is a cancer cell, e.g., a tumor cell. In some embodiments, the T cell expresses a chimeric antigen receptor (CAR) or a T cell receptor (TCR), and the target cell expresses a target antigen.

The term “genetically engineered,” “gene editing,” or “engineered” refers to a method of modifying the genome of a cell, including, but not limited to, deleting a coding or non-coding region or a portion thereof or inserting a coding region or a portion thereof. In some embodiments, the cell that is modified is a lymphocyte, e.g., a T cell, which can either be obtained from a patient or a donor. The cell can be modified to express an exogenous construct, such as, e.g., a chimeric antigen receptor (CAR) or a T cell receptor (TCR), which is incorporated into the cell's genome.

Chimeric antigen receptors (CARs or CAR-Ts) and the T cell receptors (TCRs) of the application are genetically engineered receptors. These engineered receptors may be readily inserted into and expressed by lymphocytes, including T cells, in accordance with techniques known in the art. With a CAR, a single receptor may be programmed to both recognize a specific antigen and, when bound to that antigen, activate the lymphocyte to attack and destroy the cell bearing or expressing that antigen. When these antigens exist on tumor cells, a lymphocyte that expresses the CAR may target and kill the tumor cell. In one embodiment, the cells that are prepared according to the present application is a cell having a chimeric antigen receptor (CAR), or a T cell receptor, comprising an antigen binding molecule, a costimulatory domain, and an activating domain. The costimulatory domain may comprise an extracellular domain, a transmembrane domain, and an intracellular domain. In one embodiment, the extracellular domain comprises a hinge or a truncated hinge domain.

An “immune response” refers to the action of a cell of the immune system (for example, T lymphocytes, B lymphocytes, natural killer (NK) cells, macrophages, eosinophils, mast cells, dendritic cells and neutrophils) and soluble macromolecules produced by any of these cells or the liver (including Abs, cytokines, and complement) that results in selective targeting, binding to, damage to, destruction of, and/or elimination from a vertebrate's body of invading pathogens, cells or tissues infected with pathogens, cancerous or other abnormal cells, or, in cases of autoimmunity or pathological inflammation, normal human cells or tissues.

The term “immunotherapy” refers to the treatment of a subject afflicted with, or at risk of contracting or suffering a recurrence of, a disease by a method comprising inducing, enhancing, suppressing or otherwise modifying an immune response. Examples of immunotherapy include, but are not limited to, T cell therapies. T cell therapy can include adoptive T cell therapy, tumor-infiltrating lymphocyte (TIL) immunotherapy, autologous cell therapy, engineered autologous cell therapy (eACT™), and allogeneic T cell transplantation. However, one of skill in the art would recognize that the methods of preparing T cells disclosed herein would enhance the effectiveness of any transplanted T cell therapy.

The T cells of the immunotherapy can come from any source known in the art. For example, T cells can be differentiated in vitro from a hematopoietic stem cell population, or T cells can be obtained from a donor. The donor can be a subject, e.g., a subject in need of an anti-cancer treatment. T cells can be obtained from, e.g., peripheral blood mononuclear cells, bone marrow, lymph node tissue, cord blood, thymus tissue, tissue from a site of infection, ascites, pleural effusion, spleen tissue, and tumors. In addition, the T cells can be derived from one or more T cell lines available in the art. T cells can also be obtained from a unit of blood collected from a subject using any number of techniques known to the skilled artisan, such as FICOLL™ separation and/or apheresis. T cells can also be obtained from an artificial thymic organoid (ATO) cell culture system, which replicates the human thymic environment to support efficient ex vivo differentiation of T-cells from primary and reprogrammed pluripotent stem cells.

The term “engineered Autologous Cell Therapy,” which can be abbreviated as “eACT™” also known as adoptive cell transfer, is a process by which a patient's own T cells are collected and subsequently genetically altered to recognize and target one or more antigens expressed on the cell surface of one or more specific tumor cells or malignancies. T cells can be engineered to express, for example, chimeric antigen receptors (CAR) or T cell receptor (TCR). CAR positive (+) T cells are engineered to express an extracellular single chain variable fragment (scFv) with specificity for a particular tumor antigen linked to an intracellular signaling part comprising a costimulatory domain and an activating domain. The costimulatory domain can be derived from, e.g., CD81, CD28, CTLA4, CD16, OX-40, 4-1BB/CD137, CD2, CD7, CD27, CD30, CD40, programmed death-1 (PD-1), programmed death ligand-1 (PD-L1), inducible T cell costimulator (ICOS), ICOSL, lymphocyte function-associated antigen-1 (LFA-1 (CD11a/CD18), CD3 gamma, CD3 delta, CD3 epsilon, CD247, CD276 (B7-H3), LIGHT (tumor necrosis factor superfamily member 14; TNFSF14), NKG2C, Ig alpha (CD79a, CD79b), DAP-10, Fc gamma receptor, MHC class I molecule, TNF receptor proteins, Immunoglobulin-like proteins, cytokine receptors, integrins, signaling lymphocytic activation molecules (SLAM proteins), activating NK cell receptors, BTLA, a Toll ligand receptor, ICAM-1, B7-H3, CDS, ICAM-1, GITR, BAFF-R, CS-1, GPC-3, LIGHT, HVEM (LIGHTR), KIRDS2, SLAMF7, NKp80 (KLRF1), NKp44, NKp30, NKp46, CD19, CD4, CD8, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, D11b, ITGAX, CD11c, ITGB1, CD29, ITGB2, CD18, LFA-1, ITGB7, NKG2D, TNFR2, TRANCE/RANKL, DNAM1 (CD226), SLAMF4 (CD244, 2B4), CD84, CD96 (Tactile), CEACAM1, CRT AM, Ly9 (CD229), CD160 (BY55), PSGL1, CD100 (SEMA4D), CD69, SLAMF6 (NTB-A, Ly108), SLAM (SLAMFi, CD150, IPO-3), BLAME (SLAMF8), SELPLG (CD162), LTBR, LAT, GADS, SLP-76, PAG/Cbp, CD19a, a ligand that specifically binds with CD83, or any combination thereof; The activating domain can be derived from, e.g., CD3, such as CD3 zeta, epsilon, delta, gamma, or the like. In certain embodiments, the CAR is designed to have two, three, four, or more costimulatory domains. The CAR scFv can be designed to target, for example, CD19, which is a transmembrane protein expressed by cells in the B cell lineage, including all normal B cells and B cell malignances, including but not limited to NHL, CLL, and non-T cell ALL. Example CAR+ T cell therapies and constructs are described in U.S. Patent Publication Nos. 2013/0287748, 2014/0227237, 2014/0099309, and 2014/0050708, and these references are incorporated by reference in their entirety.

A “patient” as used herein includes any human who is afflicted with a cancer (e.g., a lymphoma or a leukemia). The terms “subject” and “patient” are used interchangeably herein. The term “donor subject” refers to herein a subject whose cells are being obtained for further in vitro engineering. The donor subject can be a cancer patient that is to be treated with a population of cells generated by the methods described herein (i.e., an autologous donor), or can be an individual who donates a lymphocyte sample that, upon generation of the population of cells generated by the methods described herein, will be used to treat a different individual or cancer patient (i.e., an allogeneic donor). Those subjects who receive the cells that were prepared by the present methods can be referred to as “recipient subject.”

“Stimulation,” as used herein, refers to a primary response induced by binding of a stimulatory molecule with its cognate ligand, wherein the binding mediates a signal transduction event. A “stimulatory molecule” is a molecule on a T cell, e.g., the T cell receptor (TCR)/CD3 complex, that specifically binds with a cognate stimulatory ligand present on an antigen presenting cell. A “stimulatory ligand” is a ligand that when present on an antigen presenting cell (e.g., an artificial antigen presenting cell (aAPC), a dendritic cell, a B-cell, and the like) can specifically bind with a stimulatory molecule on a T cell, thereby mediating a primary response by the T cell, including, but not limited to, activation, initiation of an immune response, proliferation, and the like. Stimulatory ligands include, but are not limited to, an MHC Class I molecule loaded with a peptide, an anti-CD3 antibody, a super agonist anti-CD28 antibody, and a super agonist anti-CD2 antibody. An “activated” or “active,” as used herein, refers to a T cell that has been stimulated. An active T cell can be characterized by expression of one or more marker selected form CD137, CD25, CD71, CD26, CD27, CD28, CD30, CD154, CD40L, and CD134.

The term “exogenous” refers to any substance derived from an external source. For example, exogenous IL-7 or exogenous IL-21 can be obtained commercially or produced recombinantly. “Exogenous IL-7” or “exogenous IL-21,” when added in or contacted with one or more T cells, indicates that the IL-7 and/or IL-21 are not produced by the T cells. In some embodiments, the T cells prior to being mixed with exogenous IL-7 or IL-21 can contain a trace amount of IL-7 and/or IL-21 that were produced by the T cells or isolated from the subject with the T cells (i.e., endogenous IL-7 or IL-21). The one or more T cells described herein can be contacted with exogenous IL-7 and/or exogenous IL-21 through any means known in the art, including addition of isolated IL-7 and/or IL-21 to the culture, inclusion of IL-7 and/or IL-21 in the culture medium, or expression of IL-7 and/or IL-21 by one or more cells in the culture other than the one or more T cells, such as by a feeder layer.

“Treatment” or “treating” of a subject refers to any type of intervention or process performed on, or the administration of one or more T cells prepared by the present disclosure to, the subject with the objective of reversing, alleviating, ameliorating, inhibiting, slowing down or preventing the onset, progression, development, severity or recurrence of a symptom, complication or condition, or biochemical indicia associated with a disease. In one embodiment, “treatment” or “treating” includes a partial remission. In another embodiment, “treatment” or “treating” includes a complete remission.

As, used herein, the term “heterologous” means from any source other than naturally occurring sequences. For example, a heterologous sequence included as a part of a costimulatory protein having the amino acid sequence of the corresponding human costimulatory protein, is amino acids that do not naturally occur as, i.e., do not align with, the wild-type human costimulatory protein. For example, a heterologous nucleotide sequence refers to a nucleotide sequence other than that of the wild-type human costimulatory protein-encoding sequence.

An “antigen” refers to any molecule that provokes an immune response or is capable of being bound by an antibody or an antigen binding molecule. The immune response may involve either antibody production, or the activation of specific immunologically competent cells, or both. A person of skill in the art would readily understand that any macromolecule, including virtually all proteins or peptides, can serve as an antigen. An antigen can be endogenously expressed, i.e., expressed by genomic DNA, or can be recombinantly expressed. An antigen can be specific to a certain tissue, such as a cancer cell, or it can be broadly expressed. In addition, fragments of larger molecules can act as antigens. In one embodiment, antigens are tumor antigens. In one particular embodiment, the antigen is all or a fragment of BCMA, FLT3, or CLL-1.

The term “transformation” is the specific process where exogenous genetic material is directly taken up and incorporated by a cell through its cell membrane. This usually occurs when the cell is in a state of competence, which is a state where the cell can uptake exogenous material.

The terms “transduction” and “transduced” refer to the process whereby foreign DNA is introduced into a cell via viral vector (see Jones et al., “Genetics: principles and analysis,” Boston: Jones & Bartlett Publ. (1998)). In some embodiments, the vector is a retroviral vector, a DNA vector, an RNA vector, an adenoviral vector, a baculoviral vector, an Epstein Barr viral vector, a papovaviral vector, a vaccinia viral vector, a herpes simplex viral vector, an adenovirus associated vector, a lentiviral vector, or any combination thereof.

The term “transposons” are segments of DNA that can move around to different positions in the genome of a single cell. In the process, they may cause mutations and increase (or decrease) the amount of DNA in the genome of the cell, and if the cell is the precursor of a gamete, in the genomes of any descendants.

As used herein, the term “in vitro cell” refers to any cell which is cultured ex vivo. In particular, an in vitro cell can include a T cell.

As used herein, “substantially” refers to a difference of at least 70%, at least 75%, at least 80%, at least 85%, at least 90%, at least 95%, at least 99% or more as compared to a control.

A “costimulatory ligand” as used herein, includes a molecule on an antigen presenting cell that specifically binds a cognate co-stimulatory molecule on a T cell. Binding of the costimulatory ligand provides a signal that mediates a T cell response, including, but not limited to, proliferation, activation, differentiation, and the like. A costimulatory ligand induces a signal that is in addition to the primary signal provided by a stimulatory molecule, for instance, by binding of a T cell receptor (TCR)/CD3 complex with a major histocompatibility complex (MHC) molecule loaded with peptide. A co-stimulatory ligand can include, but is not limited to, 3/TR6, 4-1BB ligand, agonist or antibody that binds Toll ligand receptor, B7-1 (CD80), B7-2 (CD86), CD30 ligand, CD40, CD7, CD70, CD83, herpes virus entry mediator (HVEM), human leukocyte antigen G (HLA-G), ILT4, immunoglobulin-like transcript (ILT) 3, inducible costimulatory ligand (ICOS-L), intercellular adhesion molecule (ICAM), ligand that specifically binds with B7-H3, lymphotoxin beta receptor, MHC class I chain-related protein A (MICA), MHC class I chain-related protein B (MICB), OX40 ligand, PD-L2, or programmed death (PD) L1. A co-stimulatory ligand includes, without limitation, an antibody that specifically binds with a co-stimulatory molecule present on a T cell, such as, but not limited to, CD81, 4-1BB, B7-H3, CD2, CD27, CD28, CD30, CD40, CD7, ICOS, ligand that specifically binds with CD83, lymphocyte function-associated antigen-1 (LFA-1), natural killer cell receptor C (NKG2C), OX40, PD-1, or tumor necrosis factor superfamily member 14 (TNFSF14 or LIGHT).

A “costimulatory molecule” is a cognate binding partner on a T cell that specifically binds with a costimulatory ligand, thereby mediating a costimulatory response by the T cell, such as, but not limited to, proliferation. Costimulatory molecules include, but are not limited to, 4-1BB/CD137, B7-H3, BAFF-R, BLAME (SLAMF8), BTLA, CD 33, CD 45, CD100 (SEMA4D), CD103, CD134, CD137, CD154, CD16, CD160 (BY55), CD18, CD19, CD19a, CD2, CD22, CD247, CD27, CD276 (B7-H3), CD28, CD29, CD3 (alpha; beta; delta; epsilon; gamma; zeta), CD30, CD37, CD4, CD4, CD40, CD49a, CD49D, CD49f, CD5, CD64, CD69, CD7, CD80, CD81, CD83 ligand, CD84, CD86, CD8alpha, CD8beta, CD9, CD96 (Tactile), CD1-la, CD1-1b, CD1-1c, CD1-id, CDS, CEACAM1, CRT AM, DAP-10, DNAM1 (CD226), Fc gamma receptor, GADS, GITR, HVEM (LIGHTR), IA4, ICAM-1, ICAM-1, ICOS, Ig alpha (CD79a, CD79b), IL2R beta, IL2R gamma, IL7R alpha, integrin, ITGA4, ITGA4, ITGA6, ITGAD, ITGAE, ITGAL, ITGAM, ITGAX, ITGB2, ITGB7, ITGB1, KIRDS2, LAT, LFA-1, LFA-1, LIGHT, LIGHT (tumor necrosis factor superfamily member 14; TNFSFi4), LTBR, Ly9 (CD229), lymphocyte function-associated antigen-1 (LFA-1 (CDiia/CD18), MHC class I molecule, NKG2C, NKG2D, NKp30, NKp44, NKp46, NKp80 (KLRF1), OX40, PAG/Cbp, PD-1, PSGL1, SELPLG (CD162), signaling lymphocytic activation molecule, SLAM (SLAMFi; CD150; IPO-3), SLAMF4 (CD244; 2B4), SLAMF6 (NTB-A; Ly108), SLAMF7, SLP-76, TNF, TNFr, TNFR2, Toll ligand receptor, TRANCE/RANKL, VLA1, or VLA-6, or fragments, truncations, or combinations thereof.

A “juvenile phenotype” or “juvenile cells” as used herein, refers to less differentiated immune cells, e.g., immature immune cells. In some embodiments, juvenile phenotypes or juvenile cells refer to less differentiated T cells (defined by CCR7+ CD45RA+). In some embodiments, the juvenile T cells express CCR7+ and/or CD45RA+. In other embodiments, the juvenile T cells express CCR7+ and/or CD45RA+. In some embodiments, the juvenile T cells comprise a CAR-T phenotype.

As used herein, the term “bicistronic” refers to a single messenger RNA molecule capable of making two proteins. In some embodiments, the chimeric antigen receptor (CAR) is bicistronic.

As used herein, the term “bispecific” refers to an artificial protein that can simultaneously bind to two different types of antigens or two different epitopes on the same antigen. In some embodiments, the chimeric antigen receptor (CAR) is bispecific.

Various aspects of the disclosure are described in further detail in the following subsections.

### Methods for Preparing Genetically Engineered Lymphocytes:

The methods described herein can enhance the effectiveness of a cell therapy. In certain aspects, the cell therapy can be an adoptive T cell therapy including autologous cell therapy or allogeneic cell therapy. In certain further aspects, T cell therapy broadly comprises any method of selecting, enriching in vitro, and administering to a patient autologous or allogeneic T cells that recognize and are capable of binding tumor cells. In certain aspects, the cell therapy is a therapy utilizing lymphocytes which are not T cells, including but not limited to macrophages, neutrophils, basophils, eosinophils, granulocytes, natural killer cells (NK cells), B cells, NK-T cells, mast cells, tumor infiltrating lymphocytes (TILs), myeloid derived suppressor cells (MDSCs), and dendritic cells, which cells may be genetically engineered to express at least one CAR and which may be autologous or allogenic to a patient. Genetically engineered lymphocytes manufactured in the presence of anti-CD81, IL7, and IL21 combination are very useful for autologous therapy and also for allogenic therapy as the cells produced are more robust or proliferative when exposed to a tumor antigen as well as more juvenile and produces more cytokines, e.g., IL-2, themselves when compared to cells produced under standard manufacturing process in presence of IL-2. By comparison, cells produced in presence of exogenous IL-2 are less active, i.e., more differentiated, produce less cytokines, e.g. IL-2, themselves and expand less when contacted with a tumor antigen.

In some embodiments, the methods described herein can further comprise enriching a population of lymphocytes obtained from a donor. Enrichment of a population of lymphocytes, e.g., the one or more T cells, can be accomplished by any suitable separation method including, but not limited to, the use of a separation medium (e.g., FICOLL-PAQUE™, ROSETTESEP™ HLA Total Lymphocyte enrichment cocktail, Lymphocyte Separation Medium (LSA) (MP Biomedical Cat. No. 0850494X), or the like), cell size, shape or density separation by filtration or elutriation, immunomagnetic separation (e.g., magnetic-activated cell sorting system, MACS), fluorescent separation (e.g., fluorescence activated cell sorting system, FACS), or bead-based column separation.

Stimulation of a Population of Lymphocytes with One or More Stimulating Agents to Produce a Population of Lymphocytes:

In some embodiments, the methods described herein further comprise stimulating a population of lymphocytes with one or more stimulating agents to produce a population of activated cells under suitable conditions. Any combination of one or more suitable stimulating agents can be used to produce a population of activated lymphocytes including, but not limited to, an antibody or functional fragment thereof which targets a lymphocyte stimulatory or co-stimulatory molecule (e.g., anti-CD2 antibody, anti-CD3 antibody, anti-CD28 antibody, anti-CD81 antibody or a functional fragment thereof), or any other suitable mitogen (e.g., tetradecanoyl phorbol acetate (TPA), phytohaemagglutinin (PHA), concanavalin A (conA), lipopolysaccharide (LPS), pokeweed mitogen (PWM)), or a natural ligand to a T cell stimulatory or co-stimulatory molecule.

In some embodiments, the suitable condition for stimulating the population of lymphocytes as described herein can include a temperature, for an amount of time, and/or in the presence of a level of CO₂. In certain embodiments, the temperature for stimulation is about 34° C., about 35° C., about 36° C., about 37° C., or about 38° C. In certain embodiments, the temperature for stimulation is about 34-38° C. In certain embodiments, the temperature for stimulation is from about 35-37° C. In certain embodiments, the temperature for stimulation is from about 36-38° C. In certain embodiments, the temperature for stimulation is about 36-37° C. or about 37° C.

In some embodiments, another condition for stimulating the population of lymphocytes as described herein can include a time for stimulation. In some embodiments, the time for stimulation is about 24-72 hours. In some embodiments, the time for stimulation is about 24-36 hours, about 30-42 hours, about 36-48 hours, about 40-52 hours, about 42-54 hours, about 44-56 hours, about 46-58 hours, about 48-60 hours, about 54-66 hours, or about 60-72 hours. In one particular embodiment, the time for stimulation is about 48 hours or at least about 48 hours. In other embodiments, the time for stimulation is about 44-52 hours. In certain embodiments, the time for stimulation is about 40-44 hours, about 40-48 hours, about 40-52 hours, or about 40-56 hours.

In some embodiments, other conditions for stimulating the population of lymphocytes as described herein can include a CO₂. Level. In some embodiments, the level of CO₂ for stimulation is about 1.0-10% CO₂. In some embodiments, the level of CO₂ for stimulation is about 1.0%, about 2.0%, about 3.0%, about 4.0%, about 5.0%, about 6.0%, about 7.0%, about 8.0%, about 9.0%, or about 10.0% CO₂. In one embodiment, the level of CO₂ for stimulation is about 3-7% CO₂. In other embodiments, the level of CO₂ for stimulation is about 4-6% CO₂. In still other embodiments, the level of CO₂ for stimulation is about 4.5-5.5% CO₂. In one particular embodiment, the level of CO₂ for stimulation is about 5% CO₂.

In one embodiment, the conditions for stimulating the population of lymphocytes can comprise a temperature, for an amount of time for stimulation, and/or in the presence of a level of CO₂ in any combination. For example, the step of stimulating the population of lymphocytes can comprise stimulating the population of lymphocytes with one or more T cell stimulating agents at a temperature of about 36-38° C., for an amount of time of about 44-52 hours, and in the presence of a level of CO₂ of about 4.5-5.5% CO₂.

In some embodiments, the concentration of lymphocytes useful for the methods herein is about 0.5-10.0×10⁶ cells/mL. In certain embodiments, the concentration of lymphocytes is about 0.5-1.0×10⁶ cells/mL, about 1.0-2.0×10⁶ cells/mL, about 1.0-3.0×10⁶ cells/mL, about 1.0-4.0×10⁶ cells/mL, about 1.0-5.0×10⁶ cells/mL, about 1.0-6.0×10⁶ cells/mL, about 1.0-7.0×10⁶ cells/mL, about 1.0-8.0×10⁶ cells/mL, 1.0-9.0×10⁶ cells/mL, or about 1.0-10.0×10⁶ cells/mL. In certain embodiments, the concentration of lymphocytes is about 0.5-1.0×10⁶ cells/mL. In certain embodiments, the concentration of lymphocytes is about 1.0-2.0×10⁶ cells/mL. In certain embodiments, the concentration of lymphocytes is about 1.0-1.2×10⁶ cells/mL, about 1.0-1.4×10⁶ cells/mL, about 1.0-1.6×10⁶ cells/mL, about 1.0-1.8×10⁶ cells/mL, or about 1.0-2.0×10⁶ cells/mL. In certain embodiments, the concentration of lymphocytes is at least about 0.5×10⁶ cells/mL, at least about 0.6×10⁶ cells/mL, at least about 0.7×10⁶ cells/mL, at least about 0.8×10⁶ cells/mL, at least about 0.9×10⁶ cells/mL at least about 1.0×10⁶ cells/mL, at least about 1.1×10⁶ cells/mL, at least about 1.2×10⁶ cells/mL, at least about 1.3×10⁶ cells/mL, at least about 1.4×10⁶ cells/mL, at least about 1.5×10⁶ cells/mL, at least about 1.6×10⁶ cells/mL, at least about 1.7×10⁶ cells/mL, at least about 1.8×10⁶ cells/mL, at least about 1.9×10⁶ cells/mL, at least about 2.0×10⁶ cells/mL, at least about 4.0×10⁶ cells/mL, at least about 6.0×10⁶ cells/mL, at least about 8.0×10⁶ cells/mL, or at least about 10.0×10⁶ cells/mL.

In some embodiments an anti-CD81 antibody (or functional fragment thereof) can be used in accordance with the step of stimulating the population of lymphocytes. Any soluble or immobilized anti-CD81 antibody or functional fragment thereof can be used (e.g., clone 5A6; anti-CD81). In some aspects, the antibody can be purchased commercially from vendors known in the art including, but not limited to, Miltenyi Biotec, BD Biosciences (e.g., MACS GMP CD3 pure 1 mg/mL, Part No. 170-076-116), and eBioscience, Inc. Further, one skilled in the art would understand how to produce an anti-CD81 antibody by standard methods. In some embodiments, the one or more T cell stimulating agents that are used in accordance with the step of stimulating the population of lymphocytes include an antibody or functional fragment thereof which targets a T cell stimulatory or co-stimulatory molecule in the presence of a T cell cytokine. In one aspect, the one or more T cell stimulating agents include an anti-CD81 antibody. In certain embodiments, the T cell stimulating agent includes an anti-CD81 antibody at a concentration of from about 100 ng/mL-10 μg/mL. In certain embodiments, the concentration of anti-CD81 antibody is about 100 ng/mL, about 500 ng/mL, about 1 μg/mL, about 2 μg/mL, about 3 μg/mL, about 4 μg/mL, about 5 μg/mL, about 6 μg/mL, about 7 μg/mL, about 8 μg/mL, about 9 μg/mL or about 10 μg/mL. In one particular embodiment, the concentration of anti-CD81 antibody is about 1 μg/mL. In one particular embodiment, the concentration of anti-CD81 antibody is about 2 μg/mL. In one particular embodiment, the concentration of anti-CD81 antibody is about 3 μg/mL. In one particular embodiment, the concentration of anti-CD81 antibody is about 4 μg/mL. In one particular embodiment, the concentration of anti-CD81 antibody is about 5 μg/mL. In one particular embodiment, the concentration of anti-CD81 antibody is about 6 μg/mL. In one particular embodiment, the concentration of anti-CD81 antibody is about 7 μg/mL. In one particular embodiment, the concentration of anti-CD81 antibody is about 8 μg/mL. In one particular embodiment, the concentration of anti-CD81 antibody is about 9 μg/mL. In one particular embodiment, the concentration of anti-CD81 antibody is about 10 μg/mL.

In some embodiments an anti-CD3 antibody (or functional fragment thereof), an anti-CD28 antibody (or functional fragment thereof), or a combination of anti-CD3 and anti-CD28 antibodies can be used in accordance with the step of stimulating the population of lymphocytes. Any soluble or immobilized anti-CD2, anti-CD3 and/or anti-CD28 antibody or functional fragment thereof can be used (e.g., clone OKT3 (anti-CD3), clone 145-2C11 (anti-CD3), clone UCHT1 (anti-CD3), clone L293 (anti-CD28), clone 15E8 (anti-CD28). In some aspects, the antibodies can be purchased commercially from vendors known in the art including, but not limited to, Miltenyi Biotec, BD Biosciences (e.g., MACS GMP CD3 pure 1 mg/mL, Part No. 170-076-116), and eBioscience, Inc. Further, one skilled in the art would understand how to produce an anti-CD3 and/or anti-CD28 antibody by standard methods. In some embodiments, the one or more T cell stimulating agents that are used in accordance with the step of stimulating the population of lymphocytes include an antibody or functional fragment thereof which targets a T cell stimulatory or co-stimulatory molecule in the presence of a T cell cytokine. In one aspect, the one or more T cell stimulating agents include a soluble anti-CD28 antibody. In certain embodiments, the T cell stimulating agent includes a soluble anti-CD28 antibody at a concentration of from about 1.00 μg/mL-2.00 μg/mL. In certain embodiments, the concentration of anti-CD28 antibody is about 1.00 μg/mL, about 1.10 μg/mL, about 1.20 μg/mL, about 1.30 μg/mL, about 1.40 μg/mL, about 1.50 μg/mL, about 1.60 μg/mL, about 1.61 μg/mL, about 1.62 μg/mL, about 1.63 μg/mL, about 1.64 μg/mL, about 1.65 μg/mL, about 1.66 μg/mL, about 1.67 μg/mL, about 1.68 μg/mL, about 1.69 μg/mL, about 1.70 μg/mL, about 1.80 μg/mL, about 1.90 μg/mL or about 2.00 μg/mL. In one particular embodiment, the concentration of anti-CD28 antibody is about 1.00 μg/mL. In one particular embodiment, the concentration of anti-CD28 antibody is about 1.10 μg/mL. In one particular embodiment, the concentration of anti-CD28 antibody is about 1.20 μg/mL. In one particular embodiment, the concentration of anti-CD28 antibody is about 1.30 μg/mL. In one particular embodiment, the concentration of anti-CD28 antibody is about 1.40 μg/mL. In one particular embodiment, the concentration of anti-CD28 antibody is about 1.50 μg/mL. In one particular embodiment, the concentration of anti-CD28 antibody is about 1.60 μg/mL. In one particular embodiment, the concentration of anti-CD28 antibody is about 1.61 μg/mL. In one particular embodiment, the concentration of anti-CD28 antibody is about 1.62 μg/mL. In one particular embodiment, the concentration of anti-CD28 antibody is about 1.63 μg/mL. In one particular embodiment, the concentration of anti-CD28 antibody is about 1.64 μg/mL. In one particular embodiment, the concentration of anti-CD28 antibody is about 1.65 μg/mL. In one particular embodiment, the concentration of anti-CD28 antibody is about 1.66 μg/mL. In one particular embodiment, the concentration of anti-CD28 antibody is about 1.67 μg/mL. In one particular embodiment, the concentration of anti-CD28 antibody is about 1.68 μg/mL. In one particular embodiment, the concentration of anti-CD28 antibody is about 1.69 μg/mL. In one particular embodiment, the concentration of anti-CD28 antibody is about 1.70 μg/mL. In one aspect, the one or more T cell stimulating agents include an anti-CD3 antibody. In certain embodiments, the T cell stimulating agent includes an anti-CD3 antibody at a concentration of from about 0.50 μg/mL-2.00 μg/mL. In certain embodiments, the concentration of anti-CD3 antibody is about 0.50 μg/mL, about 0.60 μg/mL, about 0.70 μg/mL, about 0.80 μg/mL, about 0.90 μg/mL, about 1.00 μg/mL, about 1.10 μg/mL, about 1.20 μg/mL, about 1.21 μg/mL, about 1.22 μg/mL, about 1.23 μg/mL, about 1.24 μg/mL, about 1.25 μg/mL, about 1.26 μg/mL, about 1.27 μg/mL, about 1.28 μg/mL, about 1.29 μg/mL, about 1.30 μg/mL, about 1.40 μg/mL, about 1.50 μg/mL, about 1.60 μg/mL, about 1.70 μg/mL, about 1.80 μg/mL, about 1.90 μg/mL or about 2.00 μg/mL. In one particular embodiment, the concentration of anti-CD3 antibody is about 1.00 μg/mL. In one particular embodiment, the concentration of anti-CD3 antibody is about 1.10 μg/mL. In one particular embodiment, the concentration of anti-CD3 antibody is about 1.20 μg/mL. In one particular embodiment, the concentration of anti-CD3 antibody is about 1.21 μg/mL. In one particular embodiment, the concentration of anti-CD3 antibody is about 1.22 μg/mL. In one particular embodiment, the concentration of anti-CD3 antibody is about 1.23 μg/mL. In one particular embodiment, the concentration of anti-CD3 antibody is about 1.24 μg/mL. In one particular embodiment, the concentration of anti-CD3 antibody is about 1.25 μg/mL. In one particular embodiment, the concentration of anti-CD3 antibody is about 1.26 μg/mL. In one particular embodiment, the concentration of anti-CD3 antibody is about 1.27 μg/mL. In one particular embodiment, the concentration of anti-CD3 antibody is about 1.28 μg/mL. In one particular embodiment, the concentration of anti-CD3 antibody is about 1.29 μg/mL. In one particular embodiment, the concentration of anti-CD3 antibody is about 1.30 μg/mL. In an alternative embodiment, T cell activation is not needed. In such embodiment, the step of stimulating the population of lymphocytes to produce a population of activated T cells is omitted from the method, and the population of lymphocytes, which can be enriched for T lymphocytes, is transduced in accordance with the steps below.

Transduction of the Population of Activated Lymphocytes with a Viral Vector:

In some embodiments, the methods described herein can comprise transducing the population of activated T cells with a viral vector comprising a nucleic acid molecule which encodes the cell surface receptor, using a single cycle transduction to produce a population of transduced T cells. Several recombinant viruses have been used as viral vectors to deliver genetic material to a cell. Viral vectors that can be used in accordance with the transduction step can be any ecotropic or amphotropic viral vector including, but not limited to, recombinant retroviral vectors, recombinant lentiviral vectors, recombinant adenoviral vectors, and recombinant adeno-associated viral (AAV) vectors. In some embodiments, the method further comprises transducing the one or more T cells with a retrovirus. According to one aspect of this embodiment, the viral vector is grown in a suspension culture in a medium which is specific for viral vector manufacturing referred to herein as a “viral vector inoculum.” Any suitable growth media and/or supplements for growing viral vectors can be used in the viral vector inoculum in accordance with the methods described herein. According to some aspects, the viral vector inoculum is then be added to the serum-free culture media described below during the transduction step.

In some embodiments, the one or more T cells can be transduced with a retrovirus. In one embodiment, the retrovirus comprises a heterologous gene encoding a cell surface receptor. In one particular embodiment, the cell surface receptor is capable of binding an antigen on the surface of a target cell, e.g., on the surface of a tumor cell.

In some embodiments, the conditions for transducing the population of activated T cells as described herein can comprise a specific time, at a specific temperature and/or in the presence of a specific level of CO₂. In certain embodiments, the temperature for transduction is about 34° C., about 35° C., about 36° C., about 37° C., or about 38° C. In one embodiment, the temperature for transduction is about 34-38° C. In another embodiment, the temperature for transduction is from about 35-37° C. In another embodiment, the temperature for transduction is from about 36-38° C. In still another embodiment, the temperature for transduction is about 36-37° C. In one particular embodiment, the temperature for transduction is about 37° C.

In certain embodiments, the time for transduction is about 12-120 hours. In some embodiments, the time for transduction is about 12-16 hours, about 12-20 hours, about 12-24 hours, about 12-28 hours, about 12-32 hours, about 12-40 hours, about 12-50 hours, about 12-60 hours, about 12-70 hours, about 12-80 hours, about 12-90 hours, about 12-100 hours, about 12-110 hours or about 12-120 hours. In other embodiments, the time for transduction is about 20 hours or at least about 20 hours. In one embodiment, the time for transduction is about 16-24 hours. In other embodiments, the time for transduction is at least about 14 hours, at least about 16 hours, at least about 18 hours, at least about 20 hours, at least about 22 hours, at least about 24 hours, at least about 26 hours, at least about 28 hours, at least about 32 hours, at least about 40 hours, at least about 50 hours, at least about 60 hours, at least about 70 hours, at least about 80 hours, at least about 90 hours, at least about 100 hours, at least about 110 hours, or at least about 120 hours.

In certain embodiments, the level of CO₂ for transduction is about 1.0-10% CO₂. In other embodiments, the level of CO₂ for transduction is about 1.0%, about 2.0%, about 3.0%, about 4.0%, about 5.0%, about 6.0%, about 7.0%, about 8.0%, about 9.0%, or about 10.0% CO₂. In one embodiment, the level of CO₂ for transduction is about 3-7% CO₂. In another embodiment, the level of CO₂ for transduction can be about 4-6% CO₂. In another embodiment, the level of CO₂ for transduction is about 4.5-5.5% CO₂. In one particular embodiment, the level of CO₂ for transduction is about 5% CO₂.

Non-viral vectors can be loosely grouped as plasmid DNA, liposome-DNA complexes (lipoplexes), and polymer-DNA complexes (polyplexes). Oligonucleotides and their analogues, either alone or in complexes, are also an example of non-viral vector-mediated gene transfer. DNA based transposon vectors offer a mechanism for non-viral gene delivery into mammalian and human cells. These vectors work via a cut-and-paste mechanism whereby transposon DNA containing a transgene(s) of interest is integrated into chromosomal DNA by a transposase enzyme. Transposons have emerged as promising vectors for transfection that can potentially overcome some of the limitations of commonly used viral vectors. Transposons stably integrate into the target cell genome, enabling persistent expression of genes of interest.

In some embodiments, transducing the population of activated T cells as described herein can be performed for a particular time, at a specific temperature and/or in the presence of a specific level of CO₂ in any combination: a temperature of about 36-38° C., for an amount of time of about 16-24 hours, and in the presence of a level of CO₂ of about 4.5-5.5% CO₂.

### Expansion of the Population of Transduced Lymphocytes:

In some embodiments, the methods described herein can comprise expanding the population of transduced one or more lymphocytes for a particular time to produce a population of engineered lymphocytes. The predetermined time for expansion can be any suitable time which allows for the production of (i) a sufficient number of cells in the population of engineered lymphocytes for at least one dose for administering to a patient, (ii) a population of engineered lymphocytes with a favorable proportion of juvenile cells compared to a typical longer process, or (iii) both (i) and (ii). This time will depend on the cell surface receptor expressed by the lymphocytes, the vector used, the dose that is needed to have a therapeutic effect, and other variables. Thus, in some embodiments, the predetermined time for expansion can be 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, or more than 21 days. In some aspects, the time for expansion is shorter than expansion methods known in the art. For example, the predetermined time for expansion can be shorter by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, or can be shorter by more than 75%. In one aspect, the time for expansion is about 3 days, and the time from enrichment of the population of lymphocytes to producing the engineered lymphocytes is about 6 days.

In some embodiments, the conditions for expanding the population of transduced T cells can include a temperature and/or in the presence of a level of CO₂. In certain embodiments, the temperature is about 34° C., about 35° C., about 36° C., about 37° C., or about 38° C. In one embodiment, the temperature is about 34-38° C. In another embodiment, the temperature is about 35-37° C. In another embodiment, the temperature is about 36-38° C. In yet another embodiment, the temperature is about 36-37° C. In one particular embodiment the temperature is about 37° C. In certain embodiments, the level of CO₂ is 1.0-10% CO₂. In other embodiments, the level of CO₂ is about 1.0%, about 2.0%, about 3.0%, about 4.0%, about 5.0%, about 6.0%, about 7.0%, about 8.0%, about 9.0%, or about 10.0% CO₂. In one embodiment, the level of CO₂ is about 4.5-5.5% CO₂. In another embodiment, the level of CO₂ is about 5% CO₂. In other embodiments, the level of CO₂ is about 3.5%, about 4.0%, about 4.5%, about 5.0%, about 5.5%, or about 6.5% CO₂. In some embodiments, the conditions for expanding the population of transduced T cells include a temperature and/or in the presence of a level of CO₂ in any combination. For example, conditions for expanding the population of transduced T cells comprise a temperature of about 36-38° C. and in the presence of a level of CO₂ of about 4.5-5.5% CO₂.

In some embodiments, each step of the methods described herein can be performed in a closed system. In certain embodiments, the closed system is a closed bag culture system, using any suitable cell culture bags (e.g., Miltenyi Biotec MACS® GMP Cell Differentiation Bags, Origen Biomedical PermaLife Cell Culture bags). In some embodiments, the cell culture bags used in the closed bag culture system are coated with a recombinant human fibronectin fragment during the transduction step. The recombinant human fibronectin fragment can include three functional domains: a central cell-binding domain, heparin-binding domain II, and a CS1-sequence. The recombinant human fibronectin fragment can be used to increase gene efficiency of retroviral transduction of lymphocytes by aiding co-localization of target cells and viral vector. In certain embodiments, the recombinant human fibronectin fragment is RETRONECTIN® (Takara Bio, Japan). In certain embodiments, the cell culture bags are coated with recombinant human fibronectin fragment at a concentration of about 1-60 μg/mL or about 1-40 μg/mL. In other embodiments, the cell culture bags are coated with recombinant human fibronectin fragment at a concentration of about 1-20 μg/mL, 20-40 μg/mL, or 40-60 μg/mL. In some embodiments, the cell culture bags are coated with about 1 μg/mL, about 2 μg/mL, about 3 μg/mL, about 4 μg/mL, about 5 μg/mL, about 6 μg/mL, about 7 μg/mL, about 8 μg/mL, about 9 μg/mL, about 10 μg/mL, about 11 μg/mL, about 12 μg/mL, about 13 μg/mL, about 14 μg/mL, about 15 μg/mL, about 16 μg/mL, about 17 μg/mL, about 18 μg/mL, about 19 μg/mL, or about 20 μg/mL recombinant human fibronectin fragment. In other embodiments, the cell culture bags are coated with about 2-5 μg/mL, about 2-10 μg/mL, about 2-20 μg/mL, about 2-25 μg/mL, about 2-30 μg/mL, about 2-35 μg/mL, about 2-40 μg/mL, about 2-50 μg/mL, or about 2-60 μg/mL recombinant human fibronectin fragment. In certain embodiments, the cell culture bags are coated with at least about 2 μg/mL, at least about 5 μg/mL, at least about 10 μg/mL, at least about 15 μg/mL, at least about 20 μg/mL, at least about 25 μg/mL, at least about 30 μg/mL, at least about 40 μg/mL, at least about 50 μg/mL, or at least about 60 μg/mL recombinant human fibronectin fragment. In one particular embodiment, the cell culture bags are coated with at least about 10 μg/mL recombinant human fibronectin fragment. The cell culture bags used in the closed bag culture system can optionally be blocked with human albumin serum (HSA) during the transduction step. In an alternative embodiment, the cell culture bags are not blocked with HSA during the transduction step.

### T Cell Therapy:

In some embodiments, for example, and without limitation, the methods described herein can enhance the effectiveness of a T cell therapy, which can be an adoptive T cell therapy selected from the group consisting of tumor-infiltrating lymphocyte (TIL) immunotherapy, autologous cell therapy, engineered autologous cell therapy (eACT), allogeneic T cell transplantation, non-T cell transplantation, and any combination thereof. Adoptive T cell therapy broadly includes any method of selecting, enriching in vitro, and administering to a patient autologous or allogeneic T cells that recognize and are capable of binding tumor cells. TIL immunotherapy is a type of adoptive T cell therapy, wherein lymphocytes capable of infiltrating tumor tissue are isolated, enriched in vitro, and administered to a patient. The TIL cells can be either autologous or allogeneic. Autologous cell therapy is an adoptive T cell therapy that involves isolating T cells capable of targeting tumor cells from a patient, enriching the T cells in vitro, and administering the T cells back to the same patient. Allogeneic T cell transplantation can include transplant of naturally occurring T cells expanded ex vivo or genetically engineered T cells. Engineered autologous cell therapy, as described in more detail above, is an adoptive T cell therapy wherein a patient's own lymphocytes are isolated, genetically modified to express a tumor targeting molecule, expanded in vitro, and administered back to the patient. Non-T cell transplantation can include autologous or allogeneic therapies with non-T cells such as, but not limited to, natural killer (NK) cells.

In some embodiments, the one or more T cells are transduced with a retrovirus comprising a heterologous gene encoding a cell surface receptor. In one particular embodiment, the cell surface receptor is capable of binding an antigen on the surface of a target cell, e.g., on the surface of a tumor cell. In some embodiments the cell surface receptor is a chimeric antigen receptor or a T cell receptor.

In some embodiments, the one or more T cells can be engineered to express a chimeric antigen receptor. The chimeric antigen receptor can comprise a binding molecule to a tumor antigen. The binding molecule can be an antibody or an antigen binding molecule thereof. For example, the antigen binding molecule can be selected from scFv, Fab, Fab′, Fv, F(ab′)2, and dAb, and any fragments or combinations thereof.

In some embodiments, the chimeric antigen receptor can further comprise a hinge region. The hinge region can be derived from the hinge region of IgG1, IgG2, IgG3, IgG4, IgA, IgD, IgE, IgM, CD28, or CD8 alpha. In one particular embodiment, the hinge region is derived from the hinge region of IgG4.

In some embodiments, the chimeric antigen receptor can also comprise a transmembrane domain. The transmembrane domain can be a transmembrane domain of any transmembrane molecule that is a co-receptor on lymphocytes or a transmembrane domain of a member of the immunoglobulin superfamily. In certain embodiments, the transmembrane domain is derived from a transmembrane domain of CD28, CD8 alpha, CD4, or CD19. In one particular embodiment, the transmembrane domain comprises a domain derived from a CD28 transmembrane domain. In another particular embodiment, the transmembrane domain comprises a domain derived from a CD28 transmembrane domain.

In some embodiments, the chimeric antigen receptor can further comprise one or more costimulatory signaling regions. For example, the costimulatory signaling region can be a signaling region of CD28, OX-40, 41BB, CD27, inducible T cell costimulator (ICOS), CD3 gamma, CD3 delta, CD3 epsilon, CD247, Ig alpha (CD79a, CD79b), or Fc gamma receptor. In one particular embodiment, the costimulatory signaling region is a CD28 signaling region.

In one embodiment, the chimeric antigen receptor further comprises a CD3 zeta signaling domain.

In some embodiments, the chimeric antigen receptor can be engineered to target a particular tumor antigen, a “gene of interest”. In some embodiments, the tumor antigen is selected from 707-AP (707 alanine proline), AFP (alpha (a)-fetoprotein), ART-4 (adenocarcinoma antigen recognized by T4 cells), BAGE (B antigen; b-catenin/m, b-catenin/mutated), BCMA (B cell maturation antigen), Bcr-abl (breakpoint cluster region-Abelson), CAIX (carbonic anhydrase IX), CD19 (cluster of differentiation 19), CD20 (cluster of differentiation 20), CD22 (cluster of differentiation 22), CD30 (cluster of differentiation 30), CD33 (cluster of differentiation 33), CD44v7/8 (cluster of differentiation 44, exons 7/8), CAMEL (CTL-recognized antigen on melanoma), CAP-1 (carcinoembryonic antigen peptide-1), CASP-8 (caspase-8), CDC27m (cell-division cycle 27 mutated), CDK4/m (cycline-dependent kinase 4 mutated), CEA (carcinoembryonic antigen), CT (cancer/testis (antigen)), Cyp-B (cyclophilin B), DAM (differentiation antigen melanoma), EGFR (epidermal growth factor receptor), EGFRvIII (epidermal growth factor receptor, variant III), EGP-2 (epithelial glycoprotein 2), EGP-40 (epithelial glycoprotein 40), Erbb2, 3, 4 (erythroblastic leukemia viral oncogene homolog-2, -3, 4), ELF2M (elongation factor 2 mutated), ETV6-AML1 (Ets variant gene 6/acute myeloid leukemia 1 gene ETS), FBP (folate binding protein), fAchR (Fetal acetylcholine receptor), G250 (glycoprotein 250), GAGE (G antigen), GD2 (disialoganglioside 2), GD3 (disialoganglioside 3), GnT-V (N-acetylglucosaminyltransferase V), Gp100 (glycoprotein 100kD), HAGE (helicose antigen), HER-2/neu (human epidermal receptor-2/neurological; also known as EGFR2), HLA-A (human leukocyte antigen-A) HPV (human papilloma virus), HSP70-2M (heat shock protein 70-2 mutated), HST-2 (human signet ring tumor-2), hTERT or hTRT (human telomerase reverse transcriptase), iCE (intestinal carboxyl esterase), IL-13R-a2 (Interleukin-13 receptor subunit alpha-2), KIAA0205, KDR (kinase insert domain receptor), κ-light chain, LAGE (L antigen), LDLR/FUT (low density lipid receptor/GDP-L-fucose: b-D-galactosidase 2-a-Lfucosyltransferase), LeY (Lewis-Y antibody), LiCAM (L1 cell adhesion molecule), MAGE (melanoma antigen), MAGE-Ai (Melanoma-associated antigen 1), mesothelin, Murine CMV infected cells, MART-1/Melan-A (melanoma antigen recognized by T cells-1/Melanoma antigen A), MC1R (melanocortin 1 receptor), Myosin/m (myosin mutated), MUC1 (mucin 1), MUM-1, -2, -3 (melanoma ubiquitous mutated 1, 2, 3), NA88-A (NA cDNA clone of patient M88), NKG2D (Natural killer group 2, member D) ligands, NY-BR-1 (New York breast differentiation antigen 1), NY-ESO-1 (New York esophageal squamous cell carcinoma-1), oncofetal antigen (h5T4), P15 (protein 15), p190 minor bcr-abl (protein of 190KD bcr-abl), Pml/RARa (promyelocytic leukaemia/retinoic acid receptor a), PRAME (preferentially expressed antigen of melanoma), PSA (prostate-specific antigen), PSCA (Prostate stem cell antigen), PSMA (prostate-specific membrane antigen), RAGE (renal antigen), RU1 or RU2 (renal ubiquitous 1 or 2), SAGE (sarcoma antigen), SART-1 or SART-3 (squamous antigen rejecting tumor 1 or 3), SSX1, -2, -3, 4 (synovial sarcoma X1, -2, -3, -4), TAA (tumor-associated antigen), TAG-72 (Tumor-associated glycoprotein 72), TEL/AML1 (translocation Ets-family leukemia/acute myeloid leukemia 1), TPI/m (triosephosphate isomerase mutated), TRP-1 (tyrosinase related protein 1, or gp75), TRP-2 (tyrosinase related protein 2), TRP-2/INT2 (TRP-2/intron 2), VEGF-R2 (vascular endothelial growth factor receptor 2), WTi (Wilms' tumor gene), and any combination thereof. In one particular embodiment, the tumor antigen is CD19.

In some embodiments, the T cell therapy comprises administering to the patient engineered T cells expressing T cell receptor (“engineered TCR T cells”). The T cell receptor (TCR) can comprise a binding molecule to a tumor antigen. In some embodiments, the tumor antigen is selected from the group consisting of 707-AP, AFP, ART-4, BAGE, BCMA, Bcr-abl, CAIX, CD19, CD20, CD22, CD30, CD33, CD44v7/8, CAMEL, CAP-1, CASP-8, CDC27m, CDK4/m, CEA, CT, Cyp-B, DAM, EGFR, EGFRvIII, EGP-2, EGP-40, Erbb2, 3, 4, ELF2M, ETV6-AML1, FBP, fAchR, G250, GAGE, GD2, GD3, GnT-V, Gp100, HAGE, HER-2/neu, HLA-A, HPV, HSP70-2M, HST-2, hTERT or hTRT, iCE, IL-13R-a2, KIAA0205, KDR, 1-light chain, LAGE, LDLR/FUT, LeY, LiCAM, MAGE, MAGE-A1, mesothelin, Murine CMV infected cells, MART-1/Melan-A, MC1R, Myosin/m, MUC1, MUM-1, -2, -3, NA88-A, NKG2D ligands, NY-BR-1, NY-ESO-1, oncofetal antigen, P15, p190 minor bcr-abl, Pml/RARa, PRAME, PSA, PSCA, PSMA, RAGE, RU1 or RU2, SAGE, SART-1 or SART-3, SSX1, -2, -3, 4, TAA, TAG-72, TEL/AML1, TPI/m, TRP-1, TRP-2, TRP-2/INT2, VEGF-R2, WTi, and any combination thereof.

In one embodiment, the TCR comprises a binding molecule to a viral oncogene. In one particular embodiment, the viral oncogene is selected from human papilloma virus (HPV), Epstein-Barr virus (EBV), and human T-lymphotropic virus (HTLV).

In still another embodiment, the TCR comprises a binding molecule to a testicular, placental, or fetal tumor antigen. In one particular embodiment, the testicular, placental, or fetal tumor antigen is selected from the group consisting of NY-ESO-1, synovial sarcoma X breakpoint 2 (SSX2), melanoma antigen (MAGE), and any combination thereof.

In another embodiment, the TCR comprises a binding molecule to a lineage specific antigen. In one particular embodiment, the lineage specific antigen is selected from the group consisting of melanoma antigen recognized by T cells 1 (MART-1), gp100, prostate specific antigen (PSA), prostate specific membrane antigen (PSMA), prostate stem cell antigen (PSCA), and any combination thereof.

In one embodiment, the T cell therapy comprises administering to the patient engineered CAR T cells expressing a chimeric antigen receptor that binds to CD19 and further comprises a CD28 costimulatory domain and a CD3-zeta signaling region. In another embodiment the engineered CAR T cells comprises a CD81 costimulatory domain. In a particular embodiment, the T cell therapy comprises administering to a patient KTE-C19.

In one embodiment, the antigenic moieties also include, but are not limited to, an Epstein-Barr virus (EBV) antigen (e.g., EBNA-1, EBNA-2, EBNA-3, LMP-1, LMP-2), ahepatitis A virus antigen (e.g., VP1, VP2, VP3), ahepatitis B virus antigen (e.g., HBsAg, HBcAg, HBeAg), a hepatitis C viral antigen (e.g., envelope glycoproteins E1 and E2), a herpes simplex virus type 1, type 2, or type 8 (HSV1, HSV2, or HSV8) viral antigen (e.g., glycoproteins gB, gC, gC, gE, gG, gH, gI, gJ, gK, gL. gM, UL20, UL32, US43, UL45, UL49A), a cytomegalovirus (CMV) viral antigen (e.g., glycoproteins gB, gC, gC, gE, gG, gH, gI, gJ, gK, gL. gM or other envelope proteins), a human immunodeficiency virus (HIV) viral antigen (glycoproteins gp120, gp41, or p24), an influenza viral antigen (e.g., hemagglutinin (HA) or neuraminidase (NA)), a measles or mumps viral antigen, a human papillomavirus (HPV) viral antigen (e.g., L1, L2), a parainfluenza virus viral antigen, a rubella virus viral antigen, a respiratory syncytial virus (RSV) viral antigen, or a varicella-zostser virus viral antigen. In such embodiments, the cell surface receptor can be any TCR, or any CAR which recognizes any of the aforementioned viral antigens on a target virally infected cell.

In other embodiments, the antigenic moiety is associated with cells having an immune or inflammatory dysfunction. Such antigenic moieties can include, but are not limited to, myelin basic protein (MBP) myelin proteolipid protein (PLP), myelin oligodendrocyte glycoprotein (MOG), carcinoembryonic antigen (CEA), pro-insulin, glutamine decarboxylase (GAD65, GAD67), heat shock proteins (HSPs), or any other tissue specific antigen that is involved in or associated with a pathogenic autoimmune process.

In some embodiments, the methods disclosed herein can involve a T cell therapy comprising the transfer of one or more T cells to a patient. The T cells can be administered at a therapeutically effective amount. For example, a therapeutically effective amount of T cells, e.g., engineered CAR+ T cells or engineered TCR+ T cells, can be at least about 10⁴ cells, at least about 10⁵ cells, at least about 10⁶ cells, at least about 10⁷ cells, at least about 10⁸ cells, at least about 10⁹, or at least about 10¹⁰. In another embodiment, the therapeutically effective amount of the T cells, e.g., engineered CAR+ T cells or engineered TCR+ T cells, is about 10⁴ cells, about 10⁵ cells, about 10⁶ cells, about 10⁷ cells, or about 10⁸ cells. In one particular embodiment, the therapeutically effective amount of the T cells, e.g., engineered CAR+ T cells or engineered TCR+ T cells, is about 1×10⁴ cells/kg, 2×10⁴ cells/kg, 3×10⁴ cells/kg, 4×10⁴ cells/kg, 5×10⁴ cells/kg, 6×10⁴ cells/kg, 7×10⁴ cells/kg, 8×10⁴ cells/kg, 9×10⁴ cells/kg, 1×10⁵ cells/kg, 2×10⁵ cells/kg, 3×10⁵ cells/kg, 4×10⁵ cells/kg, 5×10⁵ cells/kg, 6×10⁵ cells/kg, 7×10⁵ cells/kg, 8×10⁵ cells/kg, 9×10⁵ cells/kg, 1×10⁶ cells/kg, about 2×10⁶ cells/kg, about 3×10⁶ cells/kg, about 4×10⁶ cells/kg, about 5×10⁶ cells/kg, about 6×10⁶ cells/kg, about 7×10⁶ cells/kg, about 8×10⁶ cells/kg, about 9×10⁶ cells/kg, about 1×10⁷ cells/kg, about 2×10⁷ cells/kg, about 3×10⁷ cells/kg, about 4×10⁷ cells/kg, about 5×10⁷ cells/kg, about 6×10⁷ cells/kg, about 7×10⁷ cells/kg, about 8×10⁷ cells/kg, or about 9×10⁷ cells/kg.

In some embodiments, the patient is preconditioned prior to administration of the T cell therapy. The patient can be preconditioned according to any methods known in the art, including, but not limited to, treatment with one or more chemotherapy drug and/or radiotherapy. In some embodiments, the preconditioning can include any treatment that reduces the number of endogenous lymphocytes, removes a cytokine sink, increases a serum level of one or more homeostatic cytokines or pro-inflammatory factors, enhances an effector function of T cells administered after the conditioning, enhances antigen presenting cell activation and/or availability, or any combination thereof prior to a T cell therapy. In one embodiment, the preconditioning comprises increasing a serum level of one or more cytokines in the subject.

### Compositions Comprising Lymphocytes:

In some embodiments, interleukin-7 (IL-7) is a cytokine that promotes lymphocyte homeostasis and is necessary for T cell development. Endogenous IL-7 is produced by epithelial cells in the thymus and bone marrow, and its receptor, IL-7 receptor-α (IL-7R-α) is expressed by a subset of T cells, including naive T cells and T_{CM} cells. IL-7 signaling occurs various tyrosine kinases, including the Janus kinase/signal transducer and activator of transcription (Jak/STAT) pathway, PI3K, and Src family tyrosine kinases. Any exogenous IL-7 can be used in the methods described herein. In some embodiments, the exogenous IL-7 is human IL-7. In some embodiments, the exogenous IL-7 is wild-type IL-7. In other embodiments, the exogenous IL-7 is recombinant IL-7. The IL-7 can be produced and obtained by any methods known in the art, including but not limited to isolated IL-7 from one more IL-7 producing cells or obtaining a commercially available IL-7.

In some embodiments, any concentration of IL-7 can be used in the methods described herein. For example, the present method can include contacting the one or more T cells with at least about 0.001 ng/ml IL-7, at least about 0.005 ng/ml IL-7, at least about 0.01 ng/ml IL-7, at least about 0.05 ng/ml IL-7, at least about 0.1 ng/ml IL-7, at least about 0.5 ng/ml IL-7, at least about 1.0 ng/ml IL-7, at least about 1 ng/ml IL-7, at least about 2 ng/ml IL-7, at least about 3 ng/ml IL-7, at least about 4 ng/ml IL-7, at least about 5 ng/ml IL-7, at least about 6 ng/ml IL-7, at least about 7 ng/ml IL-7, at least about 8 ng/ml IL-7, at least about 9 ng/ml IL-7, at least about 10 ng/ml IL-7, at least about 11 ng/ml IL-7, at least about 12 ng/ml IL-7, at least about 13 ng/ml IL-7, at least about 14 ng/ml IL-7, at least about 15 ng/ml IL-7, at least about 20 ng/ml IL-7, at least about 25 ng/ml IL-7, at least about 30 ng/ml IL-7, at least about 35 ng/ml IL-7, at least about 40 ng/ml IL-7, at least about 45 ng/ml IL-7, at least about 50 ng/ml IL-7, at least about 100 ng/ml IL-7, at least about 200 ng/ml IL-7, at least about 300 ng/ml IL-7, at least about 400 ng/ml IL-7, at least about 500 ng/ml IL-7, or at least about 1000 ng/ml IL-7. In one embodiment, the one or more T cells are contacted with about 0.001 to about 500 ng/ml IL-7, about 0.01 to about 100 ng/ml IL-7, about 0.1 to about 50 ng/ml IL-7, about 1 to about 10 ng/ml IL-7, about 1 to about 5 ng/ml IL-7, about 5 to about 10 ng/ml IL-7, about 3 to about 7 ng/ml IL-7, or about 4 to about 6 ng/ml IL-7. In one particular embodiment, the one or more T cells are contacted with about 5 ng/ml IL-7.

In some embodiments, interleukin-21 (IL-21) is a cytokine that promotes lymphocyte homeostasis and is necessary for T cell development. IL-21 is produced by T cells and natural killer T cells that has pleiotropic actions on a wide range of immune and non-immune cell types including, but not limited to, CD4+ and CD8+ T cells, B cells, macrophages, monocytes, and dendritic cells (DCs). Any exogenous IL-21 can be used in the methods described herein. In some embodiments, the exogenous IL-21 is human IL-21. In some embodiments, the exogenous IL-21 is wild-type IL-21. In other embodiments, the exogenous IL-21 is recombinant IL-21. The IL-21 can be produced and obtained by any methods known in the art, including but not limited to isolated IL-21 from one more IL-21 producing cells or obtaining a commercially available IL-21.

In some embodiments, any concentration of IL-21 can be used in the methods described herein. For example, the present method can include contacting the one or more T cells with at least about 0.001 ng/ml IL-21, at least about 0.005 ng/ml IL-21, at least about 0.01 ng/ml IL-21, at least about 0.05 ng/ml IL-21, at least about 0.1 ng/ml IL-21, at least about 0.5 ng/ml IL-21, at least about 1.0 ng/ml IL-21, at least about 1 ng/ml IL-21, at least about 2 ng/ml IL-21, at least about 3 ng/ml IL-21, at least about 4 ng/ml IL-21, at least about 5 ng/ml IL-21, at least about 6 ng/ml IL-21, at least about 7 ng/ml IL-21, at least about 8 ng/ml IL-21, at least about 9 ng/ml IL-21, at least about 10 ng/ml IL-21, at least about 11 ng/ml IL-21, at least about 12 ng/ml IL-21, at least about 13 ng/ml IL-21, at least about 14 ng/ml IL-21, at least about 15 ng/ml IL-21, at least about 20 ng/ml IL-21, at least about 25 ng/ml IL-21, at least about 30 ng/ml IL-21, at least about 35 ng/ml IL-21, at least about 40 ng/ml IL-21, at least about 45 ng/ml IL-21, at least about 50 ng/ml IL-21, at least about 100 ng/ml IL-21, at least about 200 ng/ml IL-21, at least about 300 ng/ml IL-21, at least about 400 ng/ml IL-21, at least about 500 ng/ml IL-21, or at least about 1000 ng/ml IL-21. In one embodiment, the one or more T cells are contacted with about 0.001 to about 500 ng/ml IL-21, about 0.01 to about 100 ng/ml IL-21, about 0.1 to about 50 ng/ml IL-21, about 1 to about 10 ng/ml IL-21, about 1 to about 5 ng/ml IL-21, about 5 to about 10 ng/ml IL-21, about 3 to about 7 ng/ml IL-21, or about 4 to about 6 ng/ml IL-21. In one particular embodiment, the one or more T cells are contacted with about 5 ng/ml IL-21.

In certain embodiments, the one or more T cells have not been and are not contacted with exogenous IL-2.

In some embodiments, the one or more T cells described herein can be obtained from any source, including, for example, a human donor. The donor can be a subject in need of an anti-cancer treatment, e.g., treatment with one T cells generated by the methods described herein (i.e., an autologous donor), or can be an individual that donates a lymphocyte sample that, upon generation of the population of cells generated by the methods described herein, will be used to treat a different individual or cancer patient (i.e., an allogeneic donor). The population of lymphocytes can be obtained from the donor by any suitable method used in the art. For example, the population of lymphocytes can be obtained by any suitable extracorporeal method, venipuncture, or other blood collection method by which a sample of blood and/or lymphocytes is obtained. In one embodiment, the population of lymphocytes is obtained by apheresis. The one or more T cells can be collected from any tissue that comprises one or more T cells, including, but not limited to, a tumor. In some embodiments, a tumor or a portion thereof is collected from a subject, and one or more T cells are isolated from the tumor tissue. Any T cell can be used in the methods disclosed herein, including any T cells suitable for a T cell therapy. For example, the one or more cells useful for the disclosure can be selected from the group consisting of tumor infiltrating lymphocytes (TIL), cytotoxic T cells, CAR T cells, engineered TCR T cells, natural killer T cells, Dendritic cells, and peripheral blood lymphocytes. In one particular embodiment, the T cells are tumor infiltrating leukocytes. In certain embodiments, the one or more T cells express CD8, e.g., are CD8+ T cells. In other embodiments, the one or more T cells express CD4, e.g., are CD4+ T cells.

### Cancer Treatment:

In some embodiments, the methods of the disclosure can be used to treat a cancer in a subject, reduce the size of a tumor, kill tumor cells, prevent tumor cell proliferation, prevent growth of a tumor, eliminate a tumor from a patient, prevent relapse of a tumor, prevent tumor metastasis, induce remission in a patient, or any combination thereof. In certain embodiments, the methods induce a complete response. In other embodiments, the methods induce a partial response.

In some embodiments, cancers that can be treated include tumors that are not vascularized, not yet substantially vascularized, or vascularized. The cancer can also include solid or non-solid tumors. In certain embodiments, the cancer can be selected from a tumor derived from acute lymphoblastic leukemia (ALL), acute myeloid leukemia (AML), adenoid cystic carcinoma, adrenocortical, carcinoma, AIDS-related cancers, anal cancer, appendix cancer, astrocytomas, atypical teratoid/rhabdoid tumor, central nervous system, B-cell leukemia, lymphoma or other B cell malignancies, basal cell carcinoma, bile duct cancer, bladder cancer, bone cancer, osteosarcoma and malignant fibrous histiocytoma, brain stem glioma, brain tumors, breast cancer, bronchial tumors, burkitt lymphoma, carcinoid tumors, central nervous system cancers, cervical cancer, chordoma, chronic lymphocytic leukemia (CLL), chronic myelogenous leukemia (CML), chronic myeloproliferative disorders, colon cancer, colorectal cancer, craniopharyngioma, cutaneous t-cell lymphoma, embryonal tumors, central nervous system, endometrial cancer, ependymoblastoma, ependymoma, esophageal cancer, esthesioneuroblastoma, ewing sarcoma family of tumors extracranial germ cell tumor, extragonadal germ cell tumor extrahepatic bile duct cancer, eye cancer fibrous histiocytoma of bone, malignant, and osteosarcoma, gallbladder cancer, gastric (stomach) cancer, gastrointestinal carcinoid tumor, gastrointestinal stromal tumors (GIST), soft tissue sarcoma, germ cell tumor, gestational trophoblastic tumor, glioma, hairy cell leukemia, head and neck cancer, heart cancer, hepatocellular (liver) cancer, histiocytosis, hodgkin lymphoma, hypopharyngeal cancer, intraocular melanoma, islet cell tumors (endocrine pancreas), kaposi sarcoma, kidney cancer, langerhans cell histiocytosis, laryngeal cancer, leukemia, lip and oral cavity cancer, liver cancer (primary), lobular carcinoma in situ (LCIS), lung cancer, lymphoma, macroglobulinemia, male breast cancer, malignant fibrous histiocytoma of bone and osteosarcoma, medulloblastoma, medulloepithelioma, melanoma, merkel cell carcinoma, mesothelioma, metastatic squamous neck cancer with occult primary midline tract carcinoma involving NUT gene, mouth cancer, multiple endocrine neoplasia syndromes, multiple myeloma/plasma cell neoplasm, mycosis fungoides, myelodysplastic syndromes, myelodysplastic/myeloproliferative neoplasms, myelogenous leukemia, chronic (CML), Myeloid leukemia, acute (AML), myeloma, multiple, myeloproliferative disorders, nasal cavity and paranasal sinus cancer, nasopharyngeal cancer, neuroblastoma, non-hodgkin lymphoma, non-small cell lung cancer, oral cancer, oral cavity cancer, oropharyngeal cancer, osteosarcoma and malignant fibrous histiocytoma of bone, ovarian cancer, pancreatic cancer, papillomatosis, paraganglioma, paranasal sinus and nasal cavity cancer, parathyroid cancer, penile cancer, pharyngeal cancer, pheochromocytoma, pineal parenchymal tumors of intermediate differentiation, pineoblastoma and supratentorial primitive neuroectodermal tumors, pituitary tumor, plasma cell neoplasm/multiple myeloma, pleuropulmonary blastoma, pregnancy and breast cancer, primary central nervous system (CNS) lymphoma, prostate cancer, rectal cancer, renal cell (kidney) cancer, renal pelvis and ureter, transitional cell cancer, retinoblastoma, rhabdomyosarcoma, salivary gland cancer, sarcoma, sézary syndrome, small cell lung cancer, small intestine cancer, soft tissue sarcoma, squamous cell carcinoma, squamous neck cancer, stomach (gastric) cancer, supratentorial primitive neuroectodermal tumors, t-cell lymphoma, cutaneous, testicular cancer, throat cancer, thymoma and thymic carcinoma, thyroid cancer, transitional cell cancer of the renal pelvis and ureter, trophoblastic tumor, ureter and renal pelvis cancer, urethral cancer, uterine cancer, uterine sarcoma, vaginal cancer, vulvar cancer, Waldenström macroglobulinemia, Wilms Tumor.

In one embodiment, the method can be used to treat a tumor, wherein the tumor is a lymphoma or a leukemia. Lymphoma and leukemia are cancers of the blood that specifically affect lymphocytes. All leukocytes in the blood originate from a single type of multipotent hematopoietic stem cell found in the bone marrow. This stem cell produces both myeloid progenitor cells and lymphoid progenitor cell, which then give rise to the various types of leukocytes found in the body. Leukocytes arising from the myeloid progenitor cells include T lymphocytes (T cells), B lymphocytes (B cells), natural killer cells, and plasma cells. Leukocytes arising from the lymphoid progenitor cells include megakaryocytes, mast cells, basophils, neutrophils, eosinophils, monocytes, and macrophages. Lymphomas and leukemias can affect one or more of these cell types in a patient.

In some embodiments, in general, lymphomas can be divided into at least two sub-groups: Hodgkin lymphoma and non-Hodgkin lymphoma. Non-Hodgkin Lymphoma (NHL) is a heterogeneous group of cancers originating in B lymphocytes, T lymphocytes or natural killer cells. In the United States, B cell lymphomas represent 80-85% of cases reported. In 2013 approximately 69,740 new cases of NHL and over 19,000 deaths related to the disease were estimated to occur. Non-Hodgkin lymphoma is the most prevalent hematological malignancy and is the seventh leading site of new cancers among men and women and account for 4% of all new cancer cases and 3% of deaths related to cancer.

In some embodiments, diffuse large B cell lymphoma (DLBCL) is the most common subtype of NHL, accounting for approximately 30% of NHL cases. There are approximately 22,000 new diagnoses of DLBCL in the United States each year. It is classified as an aggressive lymphoma with the majority of patients cured with conventional chemotherapy (NCCN guidelines NHL 2014).

In some embodiments, first line therapy for DLBCL typically includes an anthracycline-containing regimen with rituximab, such as R-CHOP (rituximab, cyclophosphamide, doxorubicin, vincristine, and prednisone), which has an objective response rate of about 80% and a complete response rate of about 50% (Coiffier 2002), with about one-third of patients have refractory disease to initial therapy or relapse after R-CHOP (Sehn 2005). For those patients who relapse after response to first line therapy, approximately 40-60% of patients can achieve a second response with additional chemotherapy. The standard of care for second-line therapy for autologous stem cell transplant (ASCT) eligible patients includes rituximab and combination chemotherapy such as R-ICE (rituximab, ifosfamide, carboplatin, and etoposide) and R-DHAP (rituximab, dexamethasone, cytarabine, and cisplatin), which each have an objective response rate of about 63% and a complete response rate of about 26% (Gisselbrecht 2010). Patients who respond to second line therapy and who are considered fit enough for transplant receive consolidation with high-dose chemotherapy and ASCT, which is curative in about half of transplanted patients (Gisselbrecht 2010). Patients who failed ASCT have a very poor prognosis and no curative options.

In some embodiments, primary mediastinal large B cell lymphoma (PMBCL) has distinct clinical, pathological, and molecular characteristics compared to DLBCL. PMBCL is thought to arise from thymic (medullary) B cells and represents approximately 3% of patients diagnosed with DLBCL. PMBCL is typically identified in the younger adult population in the fourth decade of life with a slight female predominance. Gene expression profiling suggests deregulated pathways in PMBCL overlap with Hodgkin lymphoma. Initial therapy of PMBCL generally includes anthracycline-containing regimens with rituximab, such as infusional dose-adjusted etoposide, doxorubicin, and cyclophosphamide with vincristine, prednisone, and rituximab (DA-EPOCH-R), with or without involved field radiotherapy.

In some embodiments, follicular lymphoma (FL), a B cell lymphoma, is the most common indolent (slow growing) form of NHL, accounting for approximately 20% to 30% of all NHLs. Some patients with FL will transform (TFL) histologically to DLBCL which is more aggressive and associated with a poor outcome. Histological transformation to DLBCL occurs at an annual rate of approximately 3% for 15 years with the risk of transformation continuing to drop in subsequent years. The biologic mechanism of histologic transformation is unknown. Initial treatment of TFL is influenced by prior therapies for follicular lymphoma but generally includes anthracycline-containing regimens with rituximab to eliminate the aggressive component of the disease.

In some embodiments, treatment options for relapsed/refractory PMBCL and TFL are similar to those in DLBCL. Given the low prevalence of these diseases, no large prospective randomized studies in these patient populations have been conducted. Patients with chemotherapy refractory disease have a similar or worse prognosis to those with refractory DLBCL.

In some embodiments, subjects who have refractory, aggressive NHL (e.g., DLBCL, PMBCL and TFL) have a major unmet medical need and further research with novel treatments are warranted in these populations.

Accordingly, in some embodiments, the method can be used to treat a lymphoma or a leukemia, wherein the lymphoma or leukemia is a B cell malignancy. Examples of B cell malignancies include, but are not limited to, Non-Hodgkin's Lymphomas (NHL), Small lymphocytic lymphoma (SLL/CLL), Mantle cell lymphoma (MCL), FL, Marginal zone lymphoma (MZL), Extranodal (MALT lymphoma), Nodal (Monocytoid B-cell lymphoma), Splenic, Diffuse large cell lymphoma, B cell chronic lymphocytic leukemia/lymphoma, Burkitt's lymphoma, and Lymphoblastic lymphoma. In some embodiments, the lymphoma or leukemia is selected from B-cell chronic lymphocytic leukemia/small cell lymphoma, B-cell prolymphocytic leukemia, lymphoplasmacytic lymphoma (e.g., Waldenström macroglobulinemia), splenic marginal zone lymphoma, hairy cell leukemia, plasma cell neoplasms (e.g., plasma cell myeloma (i.e., multiple myeloma), or plasmacytoma), extranodal marginal zone B cell lymphoma (e.g., MALT lymphoma), nodal marginal zone B cell lymphoma, follicular lymphoma (FL), transformed follicular lymphoma (TFL), primary cutaneous follicle center lymphoma, mantle cell lymphoma, diffuse large B cell lymphoma (DLBCL), Epstein-Barr virus-positive DLBCL, lymphomatoid granulomatosis, primary mediastinal (thymic) large B-cell lymphoma (PMBCL), Intravascular large B-cell lymphoma, ALK+ large B-cell lymphoma, plasmablastic lymphoma, primary effusion lymphoma, large B-cell lymphoma arising in HHV8-associated multicentric Castleman's disease, Burkitt lymphoma/leukemia, T-cell prolymphocytic leukemia, T-cell large granular lymphocyte leukemia, aggressive NK cell leukemia, adult T-cell leukemia/lymphoma, extranodal NK/T-cell lymphoma, enteropathy-associated T-cell lymphoma, Hepatosplenic T-cell lymphoma, blastic NK cell lymphoma, Mycosis fungoides/Sezary syndrome, Primary cutaneous anaplastic large cell lymphoma, Lymphomatoid papulosis, Peripheral T-cell lymphoma, Angioimmunoblastic T cell lymphoma, Anaplastic large cell lymphoma, B-lymphoblastic leukemia/lymphoma, B-lymphoblastic leukemia/lymphoma with recurrent genetic abnormalities, T-lymphoblastic leukemia/lymphoma, and Hodgkin lymphoma. In some embodiments, the cancer is refractory to one or more prior treatments, and/or the cancer has relapsed after one or more prior treatments.

In certain embodiments, the cancer is selected from follicular lymphoma, transformed follicular lymphoma, diffuse large B cell lymphoma, and primary mediastinal (thymic) large B-cell lymphoma. In one particular embodiment, the cancer is diffuse large B cell lymphoma.

In some embodiments, the cancer is refractory to or the cancer has relapsed following one or more of chemotherapy, radiotherapy, immunotherapy (including a T cell therapy and/or treatment with an antibody or antibody-drug conjugate), an autologous stem cell transplant, or any combination thereof. In one particular embodiment, the cancer is refractory diffuse large B cell lymphoma.

In some embodiments, the cancer is treated by administering the one or more T cells to a subject, wherein the one or more T cells have been contacted with an anti-CD81 antibody, an exogenous Interleukin-7 (IL-7) and an exogenous Interleukin-21 (IL-21). In some embodiments, the one or more T cells comprise engineered CAR cells or engineered TCR cell. In one embodiment, the engineered CAR cells or the engineered T cells treat a tumor in the subject.

In some embodiments, T-cell phenotype is assessed by CCR7 and CD45RA expression. In some embodiments, T-cell phenotype is a CAR T cell phenotype. In some embodiments, the proportion of T cells with a more juvenile phenotype (CCR7⁺ CD45RA⁺) in the apheresis material directly associates with a lower product doubling time. Among CD8 T cells, the number of CCR7⁺ CD45RA⁺ T cells is associated with durable response. In some embodiments, higher peak expansion of CAR T cells in the peripheral blood, specifically estimated as CAR cells per unit of blood volume, is associated with both objective and durable response. The number of CAR T cells in peripheral blood early after infusion is associated with clinical efficacy. (Locke et. al., Tumor burden, inflammation, and product attributes determine outcomes of axicabtagene ciloleucel in large B-cell lymphoma; Blood Advances, 13 Oct. 2020; Volume 4; Number 19).

Unless defined otherwise, all technical and scientific terms used herein have the same meaning as commonly understood by one skilled in the art to which this disclosure belongs. Although any methods and materials similar or equivalent to those described herein can also be used in the practice or testing of the present disclosure, the preferred methods and materials are now described. All publications mentioned herein are incorporated herein by reference to disclose and describe the methods and/or materials in connection with which the publications are cited.

The specific examples listed below are only illustrative and by no means limiting.

### EXAMPLES

### Example 1: Influence of Duration and Conditions of CAR-T Activation and Expansion on CAR-T Phenotype

CAR-T phenotype during manufacturing can be influenced by the duration and exact conditions of CAR-T activation and expansion. A more juvenile and less differentiated CAR-T phenotype has been shown to correlate with better clinical outcomes. The effect of different CAR-T manufacturing conditions on the product phenotype was assessed using a CD19/CD20 dual targeting CAR delivered by a lentivirus vector. The conditions tested in the initial screen using three different healthy donors were as follows:
1) Standard manufacturing where cells were activated by contacting plate bound anti-CD3 and soluble anti-CD28 antibodies and culturing in IL2 containing optimizer media. Represented as “IL2”
2) Manufacturing where cells were activated by contacting plate bound anti-CD3, soluble anti-CD28 and soluble anti-CD81 antibodies and culturing in IL2 containing optimizer media. Represented as “aCD81/IL2”
3) Manufacturing where cells were activated by contacting plate bound anti-CD3, soluble anti-CD28 antibodies and culturing in IL7 and IL21 containing optimizer media. Represented as “IL7/IL21”
4) Manufacturing where cells were activated by contacting plate bound anti-CD3, soluble anti-CD28 and soluble anti-CD81 and culturing in IL7 and IL21 containing optimizer media. Represented as “aCD81/IL7/IL21”
   Pan CD3+ cells or CD4⁺/CD8⁺ cells were isolated in-house using (Prodigy™) from leukopak obtained from AllCells™ (Alameda, CA) healthy donors and frozen down in CryoStor® cell cryopreservation media (Sigma Aldrich®). Frozen T cells were thawed, activated with plate bound MACS GMP CD3 pure (OKT3) (Miltenyl Biotec) and soluble human anti-CD28 (BD Biosciences) according to manufacturer recommendations and rested overnight in IL2 (Prometheus) or with IL7 (Peprotech) and IL21 (Peprotech). The following day cells were transduced with lentivirus vectors and cultured for about 8 days in T-Cell Media (OpTmizer™ CTS™ T-Cell Expansion Basal Medium) with Expansion Supplement, CTS Immune Cell SR, CTS Glutamax (Gibco™) supplemented with the appropriate cytokines for each of the conditions mentioned above, feeding every other day. For conditions with anti-CD81, the costimulatory antibody was added during activation step with anti-CD3 and anti-CD28. On day 8 the cells were centrifuged and frozen in CryoStor® CS5 Media (BioLife Solutions®). Cells were sampled throughout the manufacturing process on Days 0, 3, 4, 5, 6, 7, and 8, and cell phenotype was assessed using flow cytometry. All antibody staining was performed at room temperature in BD Pharmingen™ Azide containing Staining Buffer (FBS). All flow cytometry data was collected on BD FACSymphony™ A5 Cell Analyzer (BD and Company) with BD FACSDiva™ software (BD and Company and data was analyzed using FlowJo (BD and Company).

The viability of the T cells during manufacturing is shown below in Table 1:

% Viability of T cells during manufacturing

| | Donor 1 | | | | Donor 2 | | | | Donor 3 | | | |
|---|---|---|---|---|---|---|---|---|---|---|---|---|
| | | | | aCD81/ | | | | aCD81/ | | | | aCD81/ |
| | | aCD81/ | IL7/ | IL7/ | | aCD81/ | IL7/ | IL7/ | | aCD81/ | IL7/ | IL7/ |
| Days | IL2 | IL2 | IL21 | IL21 | IL2 | IL2 | IL21 | IL21 | IL2 | IL2 | IL21 | IL21 |
| 0 | 96.9 | 96.9 | 96.9 | 96.9 | 93.6 | 93.6 | 93.6 | 93.6 | 91.75 | 91.75 | 91.75 | 91.75 |
| 3 | 76.65 | 79.1 | 77.7 | 77.1 | 74 | 75 | 78.75 | 77.95 | 65.55 | 69.3 | 74.65 | 68.8 |
| 4 | 75.9 | 69.6 | 77.6 | 65.6 | 82.55 | 69.25 | 78.45 | 73.45 | 64.7 | 49.9 | 69.6 | 55.45 |
| 5 | 81.05 | 69.4 | 80.85 | 70.15 | 83.95 | 72.35 | 83.85 | 72.75 | 78.3 | 57 | 76.65 | 65.45 |
| 6 | 84.4 | 85.55 | 81.55 | 85.6 | 84.7 | 86.65 | 82.55 | 86.25 | 82.7 | 77.35 | 82.65 | 79.1 |
| 7 | 82.85 | 89.15 | 81 | 88.2 | 80.95 | 87.35 | 80.45 | 87.8 | 84.4 | 84.05 | 83.75 | 86.25 |
| 8 | 87.7 | 89.9 | 82.3 | 90.5 | 85.2 | 91.25 | 83.2 | 90.05 | 90.75 | 88.8 | 88.55 | 86.75 |

The fold expansion of the T cells during manufacturing is shown below in Table 2:

Fold expansion of T cells during manufacturing

| | Donor 1 | | | | Donor 2 | | | | Donor 3 | | | |
|---|---|---|---|---|---|---|---|---|---|---|---|---|
| | | | | aCD81/ | | | | aCD81/ | | | | aCD81/ |
| | | aCD81/ | IL7/ | IL7/ | | aCD81/ | IL7/ | IL7/ | | aCD81/ | IL7/ | IL7/ |
| Days | IL2 | IL2 | IL21 | IL21 | IL2 | IL2 | IL21 | IL21 | IL2 | IL2 | IL21 | IL21 |
| 0 | 0 | 0 | 0 | 0 | 0 | 0 | 0 | 0 | 0 | 0 | 0 | 0 |
| 3 | 1.1 | 1 | 1.19 | 0.87 | 1.4 | 0.9 | 1.2 | 0.7 | 1.1 | 0.5 | 0.9 | 0.5 |
| 4 | 2 | 1.13 | 2.2 | 0.92 | 3.32 | 1.1 | 3.5 | 0.77 | 2.28 | 0.43 | 1.81 | 0.5 |
| 5 | 4.63 | 3.04 | 4.79 | 2.63 | 6.95 | 3.2 | 7.08 | 2.46 | 5 | 1.05 | 3.87 | 1.08 |
| 6 | 11.61 | 10.36 | 9.22 | 8.09 | 13.54 | 8.52 | 11.65 | 7.37 | 11.2 | 2.45 | 8.31 | 2.59 |
| 7 | 28.59 | 34.51 | 21.17 | 20.63 | 36.4 | 27.14 | 28.32 | 25.14 | 34.46 | 5.23 | 19.8 | 5.15 |
| 8 | 35.77 | 89.74 | 30.21 | 46.61 | 31.41 | 20.06 | 33.05 | 40.62 | 39.04 | 15.46 | 27.82 | 12.45 |

The CAR expression of the T cells during manufacturing is shown below in Table 3:

CAR expression of T cells during manufacturing

| | Donor 1 | | | | Donor 2 | | | | Donor 3 | | | |
|---|---|---|---|---|---|---|---|---|---|---|---|---|
| | | | | aCD81/ | | | | aCD81/ | | | | aCD81/ |
| | | aCD81/ | IL7/ | IL7/ | | aCD81/ | IL7/ | IL7/ | | aCD81/ | IL7/ | IL7/ |
| Days | IL2 | IL2 | IL21 | IL21 | IL2 | IL2 | IL21 | IL21 | IL2 | IL2 | IL21 | IL21 |
| 0 | 0 | 0 | 0 | 0 | 0 | 0 | 0 | 0 | 0 | 0 | 0 | 0 |
| 3 | 57.1 | 47.6 | 63.1 | 55.4 | 63.8 | 45 | 72.6 | 56.9 | 74.4 | 58.7 | 78.4 | 64.8 |
| 6 | 82.2 | 74.2 | 82.2 | 79 | 83.8 | 75.3 | 82.7 | 81.3 | 81.6 | 72.9 | 79.2 | 80.2 |
| 8 | 74.2 | 74.3 | 74 | 78.6 | 84.9 | 76.2 | 85.9 | 79.9 | 76.3 | 76.2 | 74.7 | 80.2 |

Since cells in all conditions displayed healthy viability, fold expansion and CAR transduction, the phenotype of the cells was assessed using different cell surface marker combinations as shown in the tables below.

The % CD4 of the T cells during manufacturing is shown below in Table 4:

Fold expansion of T cells during manufacturing

| | Donor 1 | | | | Donor 2 | | | | Donor 3 | | | |
|---|---|---|---|---|---|---|---|---|---|---|---|---|
| | | | | aCD81/ | | | | aCD81/ | | | | aCD81/ |
| | | aCD81/ | IL7/ | IL7/ | | aCD81/ | IL7/ | IL7/ | | aCD81/ | IL7/ | IL7/ |
| Days | IL2 | IL2 | IL21 | IL21 | IL2 | IL2 | IL21 | IL21 | IL2 | IL2 | IL21 | IL21 |
| 0 | 59.2 | 59.2 | 59.2 | 59.2 | 73.3 | 73.3 | 73.3 | 73.3 | 57.4 | 57.4 | 57.4 | 57.4 |
| 3 | 33.7 | 40.4 | 39 | 41.4 | 56.2 | 49.3 | 60.3 | 53.5 | 34.7 | 33.5 | 36.1 | 37.4 |
| 6 | 37.1 | 50.5 | 39.2 | 49 | 65.7 | 70.7 | 62.5 | 70.6 | 35.9 | 44.5 | 29.1 | 41.5 |
| 8 | 33.9 | 51.3 | 38.2 | 49.9 | 72.5 | 78.7 | 68.9 | 77.6 | 36.8 | 40.3 | 30.6 | 39.9 |

The % CD8 of the T cells during manufacturing is shown below in Table 5:

Fold expansion of T cells during manufacturing

| | Donor 1 | | | | Donor 2 | | | | Donor 3 | | | |
|---|---|---|---|---|---|---|---|---|---|---|---|---|
| | | | | aCD81/ | | | | aCD81/ | | | | aCD81/ |
| | | aCD81/ | IL7/ | IL7/ | | aCD81/ | IL7/ | IL7/ | | aCD81/ | IL7/ | IL7/ |
| Days | IL2 | IL2 | IL21 | IL21 | IL2 | IL2 | IL21 | IL21 | IL2 | IL2 | IL21 | IL21 |
| 0 | 37.2 | 37.2 | 37.2 | 37.2 | 22.1 | 22.1 | 22.1 | 22.1 | 37.8 | 37.8 | 37.8 | 37.8 |
| 3 | 40.6 | 33.3 | 38.1 | 33.7 | 21.2 | 15.4 | 20.2 | 16.7 | 49.7 | 34.7 | 48.4 | 34 |
| 6 | 52.9 | 42.8 | 48.7 | 43.3 | 22.9 | 18.2 | 24.4 | 19.6 | 54.8 | 46.6 | 59.3 | 49.8 |
| 8 | 54.3 | 42.7 | 48.2 | 45.2 | 25.8 | 20.3 | 29.1 | 21.3 | 57.9 | 54.1 | 60.3 | 57.4 |

Next, the memory phenotype of the CD4 and CD8 compartment was assessed using multiple cell surface markers.

The % CCR7+ CD45RA+ of the CD4+ T cells during manufacturing is shown below in Table 6:

% CCR7+ CD45RA+ of CD4+ T cells during manufacturing

| | Donor 1 | | | | Donor 2 | | | | Donor 3 | | | |
|---|---|---|---|---|---|---|---|---|---|---|---|---|
| | | | | aCD81/ | | | | aCD81/ | | | | aCD81/ |
| | | aCD81/ | IL7/ | IL7/ | | aCD81/ | IL7/ | IL7/ | | aCD81/ | IL7/ | IL7/ |
| Days | IL2 | IL2 | IL21 | IL21 | IL2 | IL2 | IL21 | IL21 | IL2 | IL2 | IL21 | IL21 |
| 0 | 37.9 | 37.9 | 37.9 | 37.9 | 45.2 | 45.2 | 45.2 | 45.2 | 13.5 | 13.5 | 13.5 | 13.5 |
| 3 | 39.6 | 31.1 | 45.9 | 34.5 | 45.5 | 42.3 | 49.3 | 47 | 20.1 | 25.7 | 21.5 | 26.3 |
| 6 | 16.7 | 28.6 | 22.8 | 33.9 | 17.6 | 27.4 | 20.9 | 35.1 | 2.63 | 6.22 | 2.94 | 8.59 |
| 8 | 5.42 | 12 | 9.72 | 12.5 | 4.86 | 7.69 | 8.64 | 14.8 | 0.81 | 4.64 | 1.49 | 4.23 |

The % CCR7+ CD45RA+ of the CD8+ T cells during manufacturing is shown below in Table 7:

% CCR7+ CD45RA+ of CD8+ T cells during manufacturing

| | Donor 1 | | | | Donor 2 | | | | Donor 3 | | | |
|---|---|---|---|---|---|---|---|---|---|---|---|---|
| | | | | aCD81/ | | | | aCD81/ | | | | aCD81/ |
| | | aCD81/ | IL7/ | IL7/ | | aCD81/ | IL7/ | IL7/ | | aCD81/ | IL7/ | IL7/ |
| Days | IL2 | IL2 | IL21 | IL21 | IL2 | IL2 | IL21 | IL21 | IL2 | IL2 | IL21 | IL21 |
| 0 | 20 | 20 | 20 | 20 | 31.7 | 31.7 | 31.7 | 31.7 | 23.6 | 23.6 | 23.6 | 23.6 |
| 3 | 32.2 | 32 | 39.8 | 39.3 | 39.2 | 41.2 | 49 | 50.7 | 23 | 38.4 | 29.3 | 43.1 |
| 6 | 30.2 | 48.5 | 42.1 | 60.4 | 35 | 54 | 44.5 | 68.3 | 11.3 | 22.2 | 13.2 | 31.9 |
| 8 | 16.2 | 31.9 | 28.3 | 39.4 | 21.3 | 33.7 | 32.5 | 46.4 | 6.32 | 24.8 | 11 | 26.6 |

As shown in the table above, cells manufactured in a CD81/IL7/IL21 showed a higher juvenile phenotype (defined by CCR7+ CD45RA+) especially during the later days of the manufacturing. Similar results were observed using alternate cell surface markers as shown below in Table 8, 9, 10, and 11.

The % CD27+ CD28+ of the CD4+ T cells during manufacturing is shown below in Table 8:

% CD27+ CD28+ of CD4+ T cells during manufacturing

| | Donor 1 | | | | Donor 2 | | | | Donor 3 | | | |
|---|---|---|---|---|---|---|---|---|---|---|---|---|
| | | | | aCD81/ | | | | aCD81/ | | | | aCD81/ |
| | | aCD81/ | IL7/ | IL7/ | | aCD81/ | IL7/ | IL7/ | | aCD81/ | IL7/ | IL7/ |
| Days | IL2 | IL2 | IL21 | IL21 | IL2 | IL2 | IL21 | IL21 | IL2 | IL2 | IL21 | IL21 |
| 0 | 89.9 | 89.9 | 89.9 | 89.9 | 91.6 | 91.6 | 91.6 | 91.6 | 81.1 | 81.1 | 81.1 | 81.1 |
| 3 | 39 | 18.9 | 46.8 | 25.1 | 41.2 | 22 | 49.7 | 33.4 | 19.7 | 8.37 | 27 | 12.7 |
| 6 | 38.8 | 61.2 | 53 | 64.8 | 44.2 | 59 | 47.3 | 63.5 | 14.6 | 21.4 | 23.5 | 23.6 |
| 8 | 24.5 | 31.9 | 37.6 | 49.8 | 25.8 | 50.5 | 27.8 | 41.2 | 8.53 | 12.6 | 16.3 | 19.4 |

The % CD27+ CD28+ of the CD8+ T cells during manufacturing is shown below in Table 9:

% CD27+ CD28+ of CD8+ T cells during manufacturing

| | Donor 1 | | | | Donor 2 | | | | Donor 3 | | | |
|---|---|---|---|---|---|---|---|---|---|---|---|---|
| | | | | aCD81/ | | | | aCD81/ | | | | aCD81/ |
| | | aCD81/ | IL7/ | IL7/ | | aCD81/ | IL7/ | IL7/ | | aCD81/ | IL7/ | IL7/ |
| Days | IL2 | IL2 | IL21 | IL21 | IL2 | IL2 | IL21 | IL21 | IL2 | IL2 | IL21 | IL21 |
| 0 | 67.2 | 67.2 | 67.2 | 67.2 | 60.3 | 60.3 | 60.3 | 60.3 | 68.6 | 68.6 | 68.6 | 68.6 |
| 3 | 14.7 | 14.6 | 20.4 | 21.2 | 12.5 | 11.9 | 16.5 | 19.3 | 5.09 | 4.92 | 7.09 | 9.97 |
| 6 | 37.9 | 69.5 | 57.4 | 80.4 | 40.3 | 74.5 | 53.6 | 84.5 | 19.6 | 37.6 | 32.3 | 42 |
| 8 | 20.5 | 41.4 | 44.5 | 69.8 | 26.5 | 60.5 | 40.7 | 71.7 | 13.7 | 27.4 | 28.1 | 42.2 |

The % CD27+ CD28+ CCR7+ CD45RA+ of the CD4+ T cells during manufacturing is shown below in Table 10:

% CD27+ CD28+ CCR7+ CD45RA+ of CD4+ T cells during manufacturing

| | Donor 1 | | | | Donor 2 | | | | Donor 3 | | | |
|---|---|---|---|---|---|---|---|---|---|---|---|---|
| | | | | aCD81/ | | | | aCD81/ | | | | aCD81/ |
| | | aCD81/ | IL7/ | IL7/ | | aCD81/ | IL7/ | IL7/ | | aCD81/ | IL7/ | IL7/ |
| Days | IL2 | IL2 | IL21 | IL21 | IL2 | IL2 | IL21 | IL21 | IL2 | IL2 | IL21 | IL21 |
| 0 | 37.8 | 37.8 | 37.8 | 37.8 | 45 | 45 | 45 | 45 | 13.4 | 13.4 | 13.4 | 13.4 |
| 3 | 19.4 | 9.23 | 26.4 | 13 | 23.3 | 12.8 | 29.4 | 21 | 6.57 | 3.86 | 9.22 | 6.18 |
| 6 | 11.3 | 23.5 | 18.2 | 27.5 | 11.6 | 21.2 | 14.5 | 27.6 | 1.41 | 3.91 | 2.01 | 5.04 |
| 8 | 3.22 | 5.13 | 6.64 | 8.49 | 2.53 | 5.86 | 4.89 | 8.75 | 0.27 | 0.99 | 0.59 | 1.61 |

The % CD27+ CD28+ CCR7+ CD45RA+ of the CD8+ T cells during manufacturing is shown below in Table 11:

% CD27+ CD28+ CCR7+ CD45RA+ of CD8+ T cells during manufacturing

| | Donor 1 | | | | Donor 2 | | | | Donor 3 | | | |
|---|---|---|---|---|---|---|---|---|---|---|---|---|
| | | | | aCD81/ | | | | aCD81/ | | | | aCD81/ |
| | | aCD81/ | IL7/ | IL7/ | | aCD81/ | IL7/ | IL7/ | | aCD81/ | IL7/ | IL7/ |
| Days | IL2 | IL2 | IL21 | IL21 | IL2 | IL2 | IL21 | IL21 | IL2 | IL2 | IL21 | IL21 |
| 0 | 19.3 | 19.3 | 19.3 | 19.3 | 27.5 | 27.5 | 27.5 | 27.5 | 22.2 | 22.2 | 22.2 | 22.2 |
| 3 | 7.07 | 8.86 | 10.8 | 13.4 | 6.59 | 8.76 | 10.5 | 15.1 | 1.81 | 3.8 | 2.84 | 7.55 |
| 6 | 17.3 | 42.4 | 31.2 | 55.4 | 19.6 | 47.4 | 30.8 | 62.7 | 4.9 | 13.8 | 7.74 | 20.6 |
| 8 | 7.35 | 18.9 | 18.4 | 32.5 | 9.43 | 27.2 | 20.1 | 37.8 | 2.17 | 12.2 | 5.47 | 17 |

Conversely, we also observed a decrease in the effector phenotype in our product as shown in the tables below:

The % CCR7− CD45RA− of the CD4+ T cells during manufacturing is shown below in Table 12:

% CCR7− CD45RA− of CD4+ T cells during manufacturing

| | Donor 1 | | | | Donor 2 | | | | Donor 3 | | | |
|---|---|---|---|---|---|---|---|---|---|---|---|---|
| | | | | aCD81/ | | | | aCD81/ | | | | aCD81/ |
| | | aCD81/ | IL7/ | IL7/ | | aCD81/ | IL7/ | IL7/ | | aCD81/ | IL7/ | IL7/ |
| Days | IL2 | IL2 | IL21 | IL21 | IL2 | IL2 | IL21 | IL21 | IL2 | IL2 | IL21 | IL21 |
| 0 | 37.9 | 37.9 | 37.9 | 37.9 | 32.8 | 32.8 | 32.8 | 32.8 | 57 | 57 | 57 | 57 |
| 3 | 22.4 | 25.7 | 18.4 | 23.4 | 17.4 | 20.2 | 13.4 | 17.8 | 37.6 | 33.9 | 37.3 | 33.1 |
| 6 | 31.7 | 13.2 | 30.4 | 13.3 | 28.2 | 12.8 | 29.2 | 12.6 | 53 | 22.9 | 61.7 | 26 |
| 8 | 4.65 | 3.44 | 5.71 | 2.97 | 10.1 | 8.08 | 10.4 | 10.9 | 9.34 | 3.1 | 8.8 | 5.55 |

The % CCR7− CD45RA− of the CD8+ T cells during manufacturing is shown below in Table 13:

% CCR7− CD45RA− of CD8+ T cells during manufacturing

| | Donor 1 | | | | Donor 2 | | | | Donor 3 | | | |
|---|---|---|---|---|---|---|---|---|---|---|---|---|
| | | | | aCD81/ | | | | aCD81/ | | | | aCD81/ |
| | | aCD81/ | IL7/ | IL7/ | | aCD81/ | IL7/ | IL7/ | | aCD81/ | IL7/ | IL7/ |
| Days | IL2 | IL2 | IL21 | IL21 | IL2 | IL2 | IL21 | IL21 | IL2 | IL2 | IL21 | IL21 |
| 0 | 19 | 19 | 19 | 19 | 22.2 | 22.2 | 22.2 | 22.2 | 20.5 | 20.5 | 20.5 | 20.5 |
| 3 | 14.9 | 12.4 | 10.9 | 9.75 | 13.7 | 10.4 | 7.81 | 8.57 | 28 | 16.2 | 22.4 | 13.4 |
| 6 | 21.1 | 9.82 | 17.3 | 6.37 | 18.4 | 7.92 | 12.1 | 3.62 | 44.8 | 19.4 | 46.6 | 15.2 |
| 8 | 35 | 15 | 21.3 | 11.7 | 33.3 | 21.5 | 23.7 | 9.61 | 61.1 | 23.8 | 51.3 | 28.6 |

### Example 2: Functional Characterization of CAR-T Cells

As shown in Example 1, manufacturing cells in the presence of anti-CD81, IL7, and IL21 resulted in a more juvenile phenotype as compared to an IL-2 based manufacturing process. In order to perform functional characterization, CAR-T cells were manufactured using the following conditions using two different heathy donor T cells as starting material. The arms to be compared were as follows:
1) Standard manufacturing where cells were activated using plate bound anti-CD3 and soluble anti-CD28 antibodies and cultured in IL2 containing optimizer media. Represented as “IL2”
2) Manufacturing where cells were activated using plate bound anti-CD3, soluble anti-CD28 and soluble anti-CD81 and cultured in IL7 and IL21 containing optimizer media.

Represented as “aCD81/IL7/IL21” Manufactured cells from the above arms were frozen on Day 6 of manufacturing. These cells were thawed overnight in RPMI media (Gibco) with 10% FBS (Gibco), phenotyped the following day and set up in a co-culture assay with multiple target lines to assess the cytotoxicity and cytokines as read outs for the functionality of the CAR-T cells.

The % cytotoxicity of the manufactured CAR T cells against three different target lines was measured at 24 hrs. Four different effector:target ratios were tested and the results are outlined in the tables 14, 15, 16 below:

TABLE 14
% Cytotoxicity of T Cells in coculture with Nalm 6

| | Donor 1 | | | | | | Donor 2 | | | | | |
|---|---|---|---|---|---|---|---|---|---|---|---|---|
| E:T | IL2 | | | aCD81/IL7/IL21 | | | IL2 | | | aCD81/IL7/IL21 | | |
| 1:1 | 95.32 | 95.96 | 96.98 | 95.34 | 95.08 | 95.17 | 97.95 | 98.84 | 98.82 | 93.75 | 95.85 | 98.62 |
| 1:3 | 80.47 | 81.28 | 82.98 | 78.57 | 79.82 | 79.85 | 87.24 | 85.94 | 87.16 | 87.81 | 88.93 | 88.83 |
| 1:9 | 58.91 | 59.36 | 59.13 | 55.83 | 51.04 | 57.21 | 62.06 | 60.89 | 62.03 | 63.26 | 63.99 | 63.33 |
| 1:27 | 46.84 | 48.14 | 51.48 | 35.97 | 42.65 | 44.53 | 43.47 | 45.89 | 49.05 | 43.04 | 44.90 | 45.88 |

TABLE 15
% Cytotoxicity of T Cells in coculture with Raji MHC dKO

| | Donor 1 | | | | | | Donor 2 | | | | | |
|---|---|---|---|---|---|---|---|---|---|---|---|---|
| E:T | IL2 | | | aCD81/IL7/IL21 | | | IL2 | | | aCD81/IL7/IL21 | | |
| 1:1 | 45.98 | 47.55 | 46.77 | 40.53 | 43.39 | 49.05 | 70.62 | 73.00 | 74.76 | 60.82 | 65.25 | 67.22 |
| 1:3 | 18.09 | 18.56 | 17.77 | 21.97 | 24.49 | 21.40 | 45.25 | 45.66 | 44.34 | 35.28 | 38.55 | 40.10 |
| 1:9 | −4.67 | −3.27 | −3.21 | 2.20 | −0.69 | 1.25 | 23.26 | 20.25 | 18.72 | 18.66 | 17.34 | 22.31 |
| 1:27 | −12.10 | −13.37 | −10.06 | −8.95 | −10.81 | −10.67 | 13.30 | 15.95 | 14.68 | 10.82 | 13.77 | 11.63 |

TABLE 16
% Cytotoxicity of T Cells in coculture with ST486 MHC dKO

| | Donor 1 | | | | | | Donor 2 | | | | | |
|---|---|---|---|---|---|---|---|---|---|---|---|---|
| E:T | IL2 | | | aCD81/IL7/IL21 | | | IL2 | | | aCD81/IL7/IL21 | | |
| 1:1 | 99.08 | 98.88 | 98.96 | 95.55 | 97.97 | 98.00 | 98.58 | 99.53 | 99.53 | 98.91 | 99.43 | 99.72 |
| 1:3 | 83.81 | 84.80 | 87.33 | 81.97 | 79.05 | 81.01 | 91.64 | 93.47 | 91.87 | 91.61 | 92.84 | 94.26 |
| 1:9 | 56.48 | 60.56 | 62.83 | 46.58 | 53.54 | 59.22 | 59.34 | 63.06 | 60.25 | 60.14 | 65.67 | 71.18 |
| 1:27 | 40.07 | 43.62 | 41.02 | 30.67 | 31.76 | 32.07 | 35.68 | 35.61 | 42.04 | 45.83 | 48.42 | 49.42 |

As shown above there was no major difference in the % cytotoxicity between cells manufactured in IL2 vs cells manufactured in aCD81/IL7/IL21.

However, cytokine assessment of co-culture supernatants collected at 24 hours using U-Plex CAR-T cell combo 1 kit (MSD) revealed that cells grown in aCD81/IL7/IL21 secreted lower effector cytokines like Granzyme A and IFN-7 while also secreting higher IL2. These results are shown below in tables 17 and 18:

TABLE 17
Donor 1: 24 Hour Coculture Cytokine Readout (pg/ml)

| Cyto- | Nalm6 | | | | Raji MHC dKO | | | | ST486 MHC dKO | | | |
|---|---|---|---|---|---|---|---|---|---|---|---|---|
| kine | IL2 | | aCD81/IL7/IL21 | | IL2 | | aCD81/IL7/IL21 | | IL2 | | aCD81/IL7/IL21 | |
| GM- | 5496.89 | 9703.25 | 4976.24 | 7552.42 | 35070.23 | 37072.74 | 27296.95 | 27491.63 | 8160.93 | 9380.51 | 6730.86 | 7667.57 |
| CSF | | | | | | | | | | | | |
| Gran- | 1276.62 | 1838.05 | 520.11 | 761.13 | 982.53 | 963.62 | 463.70 | 507.47 | 941.28 | 1088.81 | 459.69 | 541.14 |
| zyme | | | | | | | | | | | | |
| A | | | | | | | | | | | | |
| Gran- | 26937.14 | 40455.19 | 22946.46 | 30039.67 | 79490.96 | 74687.70 | 56399.56 | 51147.45 | 38927.63 | 41459.37 | 32943.28 | 36068.50 |
| zyme | | | | | | | | | | | | |
| B | | | | | | | | | | | | |
| IFN-γ | 87597.52 | 137212.40 | 67376.34 | 97048.98 | 246884.79 | 242733.64 | 148063.59 | 146099.08 | 89891.23 | 103494.20 | 63389.83 | 72569.00 |
| IL-2 | 3159.58 | 5469.42 | 7449.45 | 11471.76 | 13256.77 | 13244.14 | 21591.32 | 20826.22 | 701.87 | 835.18 | 2035.18 | 2212.84 |
| TNF- | 1608.63 | 2734.38 | 1815.23 | 2747.45 | 3579.64 | 3719.52 | 3314.17 | 3278.56 | 851.64 | 969.79 | 826.48 | 897.60 |
| α | | | | | | | | | | | | |

TABLE 18
Donor 2: 24 Hour Coculture Cytokine Readout (pg/ml)

| Cyto- | Nalm6 | | | | Raji MHC dKO | | | | ST486 MHC dKO | | | |
|---|---|---|---|---|---|---|---|---|---|---|---|---|
| kine | IL2 | | aCD81/IL7/IL21 | | IL2 | | aCD81/IL7/IL21 | | IL2 | | aCD81/IL7/IL21 | |
| GM- | 5542.56 | 6235.43 | 5340.36 | 4913.19 | 22897.91 | 25415.45 | 19239.55 | 19333.32 | 5457.84 | 5752.32 | 5507.08 | 5926.93 |
| CSF | | | | | | | | | | | | |
| Gran- | 5979.74 | 6448.55 | 4129.18 | 3544.53 | 5925.32 | 6023.36 | 3670.01 | 3071.53 | 5592.03 | 5555.74 | 3547.49 | 3688.73 |
| zyme | | | | | | | | | | | | |
| A | | | | | | | | | | | | |
| Gran- | 25131.68 | 25234.77 | 23669.33 | 19079.02 | 53136.50 | 52023.92 | 44272.73 | 37600.28 | 30337.95 | 28310.04 | 28288.21 | 26612.28 |
| zyme | | | | | | | | | | | | |
| B | | | | | | | | | | | | |
| IFN-γ | 128148.82 | 140115.22 | 145745.77 | 128422.73 | 332726.79 | 364574.96 | 264133.75 | 266382.18 | 126607.49 | 129203.21 | 132815.75 | 134333.39 |
| IL-2 | 595.64 | 734.56 | 2421.91 | 2351.73 | 3179.36 | 3333.48 | 8461.85 | 8236.31 | 36.93 | 44.64 | 321.57 | 306.56 |
| TNF- | 1013.22 | 1137.68 | 1210.09 | 1078.12 | 2091.19 | 2286.26 | 2103.89 | 1950.86 | 467.53 | 463.50 | 462.68 | 493.82 |
| α | | | | | | | | | | | | |

Next, the ability of these cells to expand in a serial restimulation assay was evaluated. Briefly, CAR-T cells and CD19+ Nalm6 target cells from American Type Culture Company (ATCC, Manassas, VA) were incubated together at 1:1 effector:target ratio. 2 days later, a sample was collected, stained for different markers and phenotyped using flow cytometry. Absolute cell counts for both effector and target cells were also determined by including counting beads (ThermoFisher Scientific) during flow cytometry. As the CAR-T cells expanded during the assay, extra target cells were added to bring the E:T ratio back to 1:1 each time the cells were phenotyped. The assay was continued for 21 days.

The fold expansion of the manufactured CAR T cells from the co-culture are outlined below in table 19:

Fold Expansion of T Cells

| | Donor 1 | | | | Donor 2 | | | |
|---|---|---|---|---|---|---|---|---|
| Day | IL2 | | aCD81/IL7/IL21 | | IL2 | | aCD81/IL7/IL21 | |
| 0 | 1.00 | 1.00 | 1.00 | 1.00 | 1.00 | 1.00 | 1.00 | 1.00 |
| 2 | 0.61 | 0.88 | 0.78 | 0.87 | 0.63 | 0.70 | 0.97 | 1.19 |
| 4 | 1.04 | 1.58 | 1.59 | 1.97 | 0.80 | 1.09 | 1.93 | 2.33 |
| 7 | 2.29 | 4.13 | 6.15 | 9.04 | 0.96 | 1.56 | 4.51 | 5.26 |
| 9 | 2.51 | 4.69 | 8.68 | 11.07 | 0.59 | 0.99 | 4.62 | 5.10 |
| 11 | 6.84 | 13.51 | 25.93 | 35.26 | 0.64 | 1.02 | 7.69 | 7.34 |
| 14 | 21.51 | 40.53 | 44.93 | 42.96 | 0.42 | 0.45 | 8.64 | 5.88 |
| 16 | 23.89 | 37.66 | 32.22 | 32.92 | 0.11 | 0.14 | 6.50 | 3.97 |
| 18 | 20.51 | 29.01 | 29.56 | 25.84 | 0.05 | 0.03 | 1.02 | 0.68 |
| 21 | 8.74 | 18.58 | 26.19 | 25.63 | 0.05 | 0.01 | 0.05 | 0.03 |

The above results clearly show that CAR-T cell manufactured in aCD81/IL7/IL21 are superior in their ability to expand upon repeated antigen stimulation as compared to CAR-T cells manufactured in IL2.

### Example 3: In Vivo Efficacy of CAR-T Cells Manufactured in aCD81/IL7/IL21

This example describes the evaluation of the efficacy of CAR-T cells manufactured in IL2 vs CAR-T cells manufactured in aCD81/IL7/IL21 as tested in vivo in the Nalm6-luc-MHC DKO disseminated mouse model.

CD19+ Nalm6-luc-MHC DKO cells containing a bioluminescent reporter were grown in 90% RPMI, 10% FBS, 1% L-Glutamine. NSG mice (NOD.Cg-Prkdc_{scid} Il2rg_{tm1Wji}/SzJ) from Jackson Laboratory were used for the study. 8 week old mice were implanted by injecting intravenously via the lateral tail vein on day 0 with 5.0×10₅ CD19+ Nalm6-luc-MHC DKO cells in 0.1 ml using a BD U-100 Insulin Syringes ½cc, 28G. All CAR-T cells and untransduced (NTD) cells were manufactured as described in Example 1 and frozen down on Day 3 of manufacturing. 100 ul of freshly thawed CAR-T cells were dosed in mice through intravenous injection on day 7 post CD19+ Nalm6-luc-MHC DKO implantation. Three different CAR+ doses− 2e5 cells (high), 4e4 cells (medium) and 8e3 cells (low) were tested.

In vivo bioluminescence imaging was performed using an IVIS Lumina S5. Animals were imaged five at a time under ˜2-3% isoflurane gas anesthesia. Each mouse was injected IP with 150 mg/kg D-luciferin and imaged in the prone 15 minutes after the injection. Large binning of the CCD chip was used, and the exposure time was adjusted to 15 seconds to obtain at least several hundred counts from the metastatic tumors that were observable in each mouse in the image and to avoid saturation of the CCD chip. BLI images were collected on days 5, 8, 12, 15, 19, 22, 26, 29, 33, 36, 40, 44, 48, 51, 55, 58, 61 and 65. Images were analyzed using the Living Image version 4.5.4 software. Whole body fixed-volume ROIs were placed on prone images for each individual animal. Total flux (photons/sec) was calculated and exported for all ROIs.

BLI (Bioluminescence imaging) values (shown as Mean±SEM) corresponding to CD19+ Nalm6-luc-MHC DKO tumor burden in mice is presented for different treatment groups (Tables 20A-20E). Higher values indicate higher tumor burden. While both IL2 and aCD81/IL7/IL21 manufactured cells demonstrated comparable in vivo efficacy at high and medium doses, CAR-T cells manufactured with aCD81/IL7/IL21 demonstrated superior tumor control kinetics over at the lowest dose over the course of the study. In comparison, mice treated with vehicle only or untransduced cells (NTD IL2 and NTD aCD81/IL7/IL21) did not show any tumor control as expected.

TABLE 20A

| Days post tumor | | | | | | | | | | |
|---|---|---|---|---|---|---|---|---|---|---|
| inoculation | Vehicle | | | | | NTD (IL2) | | | | |
| 5 | 1.27E+07 | 6.11E+06 | 6.06E+06 | 4.56E+06 | 4.53E+06 | 1.23E+07 | 6.14E+06 | 5.75E+06 | 4.71E+06 | 4.50E+06 |
| 8 | 1.28E+08 | 5.72E+07 | 6.38E+07 | 4.31E+07 | 6.22E+07 | 1.30E+08 | 5.09E+06 | 5.66E+07 | 5.70E+07 | 4.59E+07 |
| 12 | 5.42E+09 | 4.00E+09 | 2.38E+09 | 2.60E+09 | 2.13E+09 | 6.18E+09 | 3.42E+09 | 3.20E+09 | 2.99E+09 | 2.62E+09 |
| 15 | 1.39E+10 | 8.68E+09 | 9.48E+09 | 9.49E+09 | 9.04E+09 | 1.56E+10 | 1.35E+10 | 1.57E+10 | 1.60E+10 | 1.08E+10 |
| 19 | 2.23E+10 | 2.80E+10 | 8.25E+08 | 2.86E+10 | 2.57E+10 | 3.26E+10 | 2.86E+10 | 3.02E+10 | 3.48E+10 | 3.27E+10 |
| 22 | 4.47E+10 | 4.17E+10 | 4.59E+10 | 3.55E+10 | 3.92E+10 | 4.75E+10 | 4.82E+10 | 5.80E+10 | 6.61E+10 | 4.67E+10 |
| 26 | | | | | | | | | | |
| 29 | | | | | | | | | | |
| 33 | | | | | | | | | | |
| 36 | | | | | | | | | | |
| 40 | | | | | | | | | | |
| 44 | | | | | | | | | | |
| 48 | | | | | | | | | | |
| 51 | | | | | | | | | | |
| 55 | | | | | | | | | | |
| 58 | | | | | | | | | | |
| 61 | | | | | | | | | | |
| 65 | | | | | | | | | | |

TABLE 20B

| Days post tumor | | | | | | | | | | |
|---|---|---|---|---|---|---|---|---|---|---|
| inoculation | NTD (aCD81/IL7/IL21) | | | | | CAR (IL2) 2e5 | | | | |
| 5 | 1.04E+07 | 6.24E+06 | 5.75E+06 | 4.74E+06 | 4.43E+06 | 8.26E+06 | 6.56E+06 | 5.62E+06 | 4.84E+06 | 4.09E+06 |
| 8 | 9.78E+07 | 9.34E+07 | 6.05E+07 | 5.68E+07 | 4.52E+07 | 9.42E+07 | 7.78E+07 | 7.66E+07 | 8.78E+07 | 6.56E+07 |
| 12 | 4.63E+09 | 4.65E+09 | 3.12E+09 | 2.44E+09 | 2.06E+09 | 1.18E+08 | 1.97E+08 | 1.52E+08 | 1.50E+08 | 2.18E+08 |
| 15 | 1.13E+10 | 1.37E+10 | 1.12E+10 | 8.98E+09 | 9.93E+09 | 4.28E+06 | 4.36E+06 | 1.10E+07 | 5.90E+06 | 1.67E+07 |
| 19 | 2.86E+10 | 2.96E+10 | 2.94E+10 | 2.29E+10 | 2.58E+10 | 7.96E+05 | 9.64E+05 | 9.66E+05 | 7.64E+05 | 9.74E+05 |
| 22 | 3.85E+10 | 5.66E+10 | 3.92E+10 | 3.74E+10 | 4.89E+10 | 6.41E+05 | 6.28E+05 | 7.37E+05 | 6.58E+05 | 7.24E+05 |
| 26 | | | | | | 6.10E+05 | 7.45E+05 | 7.47E+05 | 6.72E+05 | 7.17E+05 |
| 29 | | | | | | 6.42E+05 | 7.91E+05 | 1.19E+06 | 8.19E+05 | 8.23E+05 |
| 33 | | | | | | 6.40E+05 | 7.79E+05 | 1.24E+06 | 9.26E+05 | 9.22E+05 |
| 36 | | | | | | 6.50E+05 | 8.02E+05 | 1.43E+06 | 9.25E+05 | 8.02E+05 |
| 40 | | | | | | 6.77E+05 | 7.81E+05 | 1.72E+06 | 8.67E+05 | 9.19E+05 |
| 44 | | | | | | 8.32E+05 | 8.66E+05 | 3.04E+06 | 9.40E+05 | 1.09E+06 |
| 48 | | | | | | 7.81E+05 | 8.16E+05 | 2.33E+06 | 7.85E+05 | 9.18E+05 |
| 51 | | | | | | 8.04E+05 | 1.27E+06 | 6.47E+07 | 3.49E+06 | 9.71E+05 |
| 55 | | | | | | 1.72E+06 | 4.65E+06 | 4.89E+08 | 1.60E+07 | 1.90E+06 |
| 58 | | | | | | 1.20E+06 | 3.62E+06 | 3.83E+08 | 6.81E+06 | 1.13E+06 |
| 61 | | | | | | 1.60E+07 | 1.31E+08 | 8.78E+09 | 2.15E+08 | 1.48E+07 |
| 65 | | | | | | 3.97E+07 | 4.56E+08 | 1.20E+10 | 3.29E+08 | 2.92E+07 |

TABLE 20C

| Days post tumor | | | | | | | | | | |
|---|---|---|---|---|---|---|---|---|---|---|
| inoculation | CAR (aCD81/IL7/IL21) 2e5 | | | | | CAR (IL2) 4e4 | | | | |
| 5 | 7.44E+06 | 6.89E+06 | 5.54E+06 | 5.20E+06 | 3.15E+06 | 8.14E+06 | 6.59E+06 | 5.60E+06 | 5.08E+06 | 3.67E+06 |
| 8 | 3.51E+06 | 1.09E+08 | 7.78E+07 | 8.01E+07 | 3.66E+07 | 4.50E+06 | 6.44E+07 | 8.50E+07 | 5.81E+07 | 4.03E+06 |
| 12 | 1.48E+08 | 5.19E+07 | 3.35E+07 | 3.98E+07 | 1.53E+07 | 2.33E+09 | 2.40E+09 | 1.86E+09 | 1.59E+09 | 1.18E+09 |
| 15 | 6.97E+05 | 1.22E+06 | 2.74E+06 | 1.60E+06 | 3.08E+06 | 3.07E+09 | 7.27E+09 | 3.02E+09 | 2.10E+09 | 1.48E+09 |
| 19 | 7.49E+05 | 7.85E+05 | 6.51E+05 | 7.96E+05 | 7.85E+05 | 1.02E+06 | 1.14E+06 | 8.76E+05 | 1.07E+06 | 8.67E+05 |
| 22 | 8.39E+05 | 9.03E+05 | 8.95E+05 | 8.10E+05 | 7.74E+05 | 8.96E+05 | 9.88E+05 | 1.02E+06 | 8.63E+05 | 6.80E+05 |
| 26 | 5.65E+05 | 7.11E+05 | 7.65E+05 | 6.84E+05 | 7.85E+05 | 6.46E+05 | 9.76E+05 | 7.79E+05 | 6.63E+05 | 7.98E+05 |
| 29 | 8.98E+05 | 7.71E+05 | 1.00E+06 | 8.28E+05 | 8.95E+05 | 6.71E+05 | 5.74E+05 | 6.93E+05 | 6.35E+05 | 6.12E+05 |
| 33 | 6.00E+05 | 5.87E+05 | 1.38E+06 | 6.84E+05 | 6.54E+05 | 8.45E+05 | 1.01E+06 | 8.17E+05 | 9.12E+05 | 8.15E+05 |
| 36 | 8.68E+05 | 8.43E+05 | 1.26E+06 | 8.53E+05 | 8.57E+05 | 8.75E+05 | 7.00E+05 | 1.01E+06 | 8.68E+05 | 9.22E+05 |
| 40 | 6.76E+05 | 7.11E+05 | 1.07E+06 | 7.15E+05 | 9.86E+05 | 7.64E+05 | 1.35E+06 | 1.02E+06 | 8.80E+05 | 9.03E+05 |
| 44 | 1.20E+06 | 1.13E+06 | 1.27E+07 | 9.61E+05 | 1.22E+06 | 9.17E+05 | 1.30E+06 | 1.07E+06 | 1.04E+06 | 9.77E+05 |
| 48 | 1.35E+06 | 1.17E+06 | 1.45E+07 | 1.15E+06 | 1.04E+06 | 8.42E+05 | 3.24E+06 | 1.12E+06 | 1.88E+06 | 9.78E+05 |
| 51 | 1.01E+06 | 8.74E+05 | 1.72E+06 | 9.09E+05 | 8.91E+05 | 9.25E+05 | 9.93E+05 | 1.14E+06 | 3.96E+06 | 1.26E+06 |
| 55 | 1.69E+06 | 2.02E+06 | 7.09E+07 | 1.69E+06 | 1.34E+06 | 8.83E+05 | 5.95E+06 | 1.17E+06 | 6.44E+05 | 7.58E+05 |
| 58 | 1.37E+06 | 2.43E+06 | 2.47E+08 | 3.26E+06 | 1.19E+06 | 1.05E+06 | 3.07E+06 | 1.38E+06 | 1.38E+06 | 7.95E+05 |
| 61 | 2.21E+06 | 6.02E+06 | 7.32E+08 | 7.65E+06 | 2.03E+06 | 2.14E+06 | 1.14E+08 | 5.43E+06 | 4.49E+06 | 1.38E+06 |
| 65 | 2.95E+06 | 9.27E+06 | 9.83E+08 | 9.29E+06 | 2.69E+06 | 1.19E+07 | 1.05E+09 | 2.37E+07 | 1.94E+07 | 2.26E+06 |

TABLE 20D

| Days post tumor | | | | | | | | | | |
|---|---|---|---|---|---|---|---|---|---|---|
| inoculation | CAR (aCD81/IL7/IL21) 4e4 | | | | | CAR (IL2) 8e3 | | | | |
| 5 | 7.40E+06 | 6.94E+06 | 5.46E+06 | 5.26E+06 | 3.00E+06 | 7.75E+06 | 6.60E+06 | 5.54E+06 | 5.14E+06 | 3.44E+06 |
| 8 | 1.37E+08 | 1.03E+08 | 7.31E+07 | 8.14E+07 | 5.26E+07 | 3.90E+06 | 7.80E+07 | 7.16E+07 | 5.36E+07 | 2.84E+06 |
| 12 | 1.81E+09 | 9.22E+08 | 1.16E+09 | 8.44E+08 | 9.34E+08 | 4.03E+09 | 3.73E+09 | 2.73E+09 | 2.22E+09 | 4.23E+09 |
| 15 | 9.10E+08 | 3.92E+08 | 6.76E+08 | 3.16E+08 | 7.77E+08 | 1.41E+10 | 9.71E+09 | 1.04E+10 | 7.87E+09 | 1.30E+10 |
| 19 | 7.78E+05 | 1.03E+06 | 1.07E+06 | 9.99E+05 | 9.04E+05 | 2.18E+10 | 1.99E+10 | 2.18E+10 | 1.64E+10 | 2.03E+10 |
| 22 | 7.31E+05 | 8.44E+05 | 8.48E+05 | 7.51E+05 | 7.76E+05 | 2.51E+10 | 2.78E+10 | 3.45E+10 | 1.24E+10 | 1.84E+10 |
| 26 | 7.18E+05 | 8.39E+05 | 7.42E+05 | 7.59E+05 | 6.15E+05 | 2.21E+10 | 4.28E+10 | 4.11E+10 | 2.01E+10 | 1.27E+10 |
| 29 | 5.64E+05 | 8.00E+05 | 7.99E+05 | 8.65E+05 | 8.43E+05 | 3.01E+10 | 4.71E+10 | 8.67E+10 | 2.14E+10 | 9.90E+09 |
| 33 | 1.41E+06 | 9.26E+05 | 8.95E+05 | 9.14E+05 | 8.41E+05 | | | | | 2.64E+10 |
| 36 | 1.33E+06 | 9.21E+05 | 8.57E+05 | 8.43E+05 | 9.18E+05 | | | | | |
| 40 | 1.58E+06 | 9.67E+05 | 8.44E+05 | 7.87E+05 | 7.75E+05 | | | | | |
| 44 | 8.76E+05 | 9.10E+05 | 6.53E+05 | 9.11E+05 | 9.86E+05 | | | | | |
| 48 | 9.28E+06 | 9.37E+05 | 8.58E+05 | 9.68E+05 | 9.94E+05 | | | | | |
| 51 | 1.59E+06 | 9.51E+05 | 9.28E+05 | 6.35E+05 | 1.01E+06 | | | | | |
| 55 | 7.50E+07 | 1.72E+06 | 8.33E+05 | 8.48E+05 | 1.15E+06 | | | | | |
| 58 | 3.17E+06 | 1.02E+06 | 9.61E+05 | 8.73E+05 | 1.08E+06 | | | | | |
| 61 | 4.62E+08 | 1.30E+07 | 1.21E+06 | 1.27E+06 | 1.56E+06 | | | | | |
| 65 | 1.68E+09 | 2.65E+07 | 2.04E+06 | 1.58E+06 | 1.90E+06 | | | | | |

TABLE 20E

| Days post | | | | | |
|---|---|---|---|---|---|
| tumor | | | | | |
| inoculation | CAR (aCD81/IL7/IL21) 8e3 | | | | |
| 5 | 7.14E+06 | 7.03E+06 | 5.42E+06 | 5.35E+06 | 2.37E+06 |
| 8 | 1.43E+08 | 1.26E+08 | 8.73E+07 | 8.72E+07 | 3.31E+07 |
| 12 | 4.31E+09 | 3.46E+09 | 3.76E+09 | 3.16E+09 | 1.88E+09 |
| 15 | 1.17E+10 | 9.76E+09 | 7.62E+09 | 9.94E+09 | 5.86E+09 |
| 19 | 6.77E+08 | 6.74E+08 | 1.15E+10 | 1.37E+10 | 7.88E+09 |
| 22 | 2.37E+07 | 2.23E+07 | 6.69E+08 | 2.58E+10 | 2.00E+09 |
| 26 | 2.25E+07 | 1.95E+07 | 2.84E+08 | 2.63E+10 | 3.03E+08 |
| 29 | 2.96E+07 | 2.24E+07 | 4.11E+08 | 3.79E+10 | 6.10E+08 |
| 33 | 4.80E+07 | 3.65E+07 | 9.67E+08 | 5.71E+10 | 1.14E+09 |
| 36 | 8.22E+05 | 2.55E+06 | 9.38E+05 | | 2.89E+06 |
| 40 | 1.01E+06 | 4.37E+06 | 1.12E+06 | | 1.84E+06 |
| 44 | 1.06E+06 | 2.20E+06 | 8.39E+05 | | 1.10E+07 |
| 48 | 1.35E+06 | 6.43E+06 | 1.33E+06 | | 7.12E+07 |
| 51 | 1.21E+06 | 1.51E+07 | 1.06E+06 | | 3.56E+06 |
| 55 | 2.02E+06 | 5.73E+07 | 2.28E+06 | | 3.29E+08 |
| 58 | 3.12E+06 | 4.57E+07 | 5.25E+06 | | 2.77E+08 |
| 61 | 4.64E+07 | 2.74E+08 | 2.43E+07 | | 1.99E+09 |
| 65 | 3.59E+07 | 3.17E+09 | 1.27E+08 | | 4.01E+09 |

While a number of embodiments have been described, it is apparent that the disclosure and examples may provide other embodiments that utilize or are encompassed by the compositions and methods described herein. Therefore, it will be appreciated that the scope of is to be defined by that which may be understood from the disclosure and the appended claims rather than by the embodiments that have been represented by way of example.

1. A method of manufacturing a genetically engineered lymphocyte comprising:
contacting in vitro one or more lymphocytes from a subject with an anti-CD81 antibody, an exogenous Interleukin-7 (IL-7) and an exogenous Interleukin-21 (IL-21);
transforming the contacted lymphocyte with a vector containing a gene of interest; and
harvesting the lymphocyte.

2. The method of claim 1, wherein the lymphocyte is selected from the group consisting of macrophages, neutrophils, basophils, eosinophils, granulocytes, natural killer cells (NK cells), B cells, T cells, NK-T cells, mast cells, tumor infiltrating lymphocytes (TILs), myeloid derived suppressor cells (MDSCs), and dendritic cells.

3. The method of claim 2, wherein the lymphocyte is a T cell.

4. The method of claim 1, wherein the lymphocyte is contacted with an anti-CD3 antibody and an anti-CD28 antibody.

5. The method of claim 3, wherein the T cell comprises CD8+ T cells and CD4+ T cells.

6. The method of claim 5, wherein the CD8+ T cells express CCR7+ CD45RA+.

7. The method of claim 5, wherein the CD4+ T cells express CCR7+ CD45RA+.

8. The method of claim 5, wherein the CD8+ T cells express CD27+ CD28+.

9. The method of claim 5, wherein the CD4+ T cells express CD27+ CD28+.

10. The method of claim 5, wherein the CD8+ T cells express CD27+ CD28+ CCR7+CD45RA+.

11. The method of claim 5, wherein the CD4+ T cells express CD27+ CD28+ CCR7+CD45RA+.

12. The method of claim 1, wherein the lymphocyte express a chimeric antigen receptor (CAR), further wherein the lymphocyte is transformed with a vector encoding the chimeric antigen receptor (CAR).

13. The method of claim 12, wherein the chimeric antigen receptor (CAR) is bispecific.

14. (canceled)

15. The method of claim 12, wherein the chimeric antigen receptor (CAR) comprises a single chain variable fragment (scFv) targeting a tumor antigen selected from the group consisting of CD19, CD20, BCMA, CLL-1, CTLA4, CD30, CD40, NKp44, NKp30, GPC-3, CD79a, CD79b, BAFF-R, CS-1, PSMA, NKG2D, CLL-1, CD33, CD22 and NKp46.

16. The method of claim 1, wherein the vector is a retroviral vector, a DNA vector, a plasmid, an RNA vector, an adenoviral vector, an adenovirus associated vector, a lentiviral vector, or any combination thereof.

17. The method of claim 16, wherein the DNA vector is a transposon.

18. The method of claim 1, wherein the lymphocyte has not been contacted with an exogenous Interleukin-2 (IL-2).

19. The method of claim 1, wherein the subject is a cancer patient.

20. (canceled)

21. The method of claim 1, wherein the vector is a lentiviral vector.

22. The method of claim 1, wherein the vector is a retroviral vector.

23. The method of claim 1, wherein the lymphocyte is harvested no more than 24 hours after transformation.

24-66. (canceled)
